(19) United States
(12) Patent Application Publication
(10) Pub. No.: US 2026/0207607 A1
(43) Pub. Date: Jul. 23, 2026

(54) COMBINATIONS OF NANOPARTICLE-LOADED IMMUNE AGONISTS AND RAS TARGETED THERAPIES FOR TREATMENT OF CANCER

(71) Applicant: UNIVERSITY OF MASSACHUSETTS, Westborough, MA (US)

(72) Inventors: Marcus RUSCETTI, Marlborough, MA (US); Loretah CHIBAYA, Worcester, MA (US); Prabhani ATUKORALE, Rutland, MA (US); Griffin KANE, Keene, NH (US); Kelly D. DEMARCO, Westborough, MA (US); Haruka MORI, Westborough, MA (US)

(21) Appl. No.: 19/143,083

(22) PCT Filed: May 13, 2024
(86) PCT No.: PCT/US2024/029125

§ 371 Date: Jun. 25, 2025

Related U.S. Application Data
(60) Provisional application No. 63/466,164, filed on May 12, 2023.

(51) Int. Cl. A61K 31/519; A61K 9/51; A61P 35/00

(52) U.S. Cl. CPC A61K 31/519; A61K 9/5123; A61P 35/00

(57) ABSTRACT

Described herein is the combination of STING/TLR4 agonist nanoparticles (NPs) and a combination of a MEK inhibitor (e.g., trametinib) and CDK4/6 inhibitor (e.g., palbociclib) treatment. This combination synergizes to (a) lead to enhanced NP uptake in multiple cell types in the TME, (b) increased antigen presentation on tumors cells as well as APCs, and (c) CD8+ T cell mediated long-term tumor control that is interferon signaling dependent. Also provided are methods to treat cancer, e.g., KRAS mutant cancers, e.g., KRAS mutant PDAC or lung cancer.

### CLAIM OF PRIORITY

This application claims the benefit of U.S. Provisional Patent Application Ser. No. 63/466,164, filed on May 12, 2023. The entire contents of the foregoing are hereby incorporated by reference.

### FEDERALLY SPONSORED RESEARCH OR DEVELOPMENT

This invention was made with Government support under Grant Nos. CA262355 and CA241110 awarded by the National Institutes of Health. The Government has certain rights in the invention.

### TECHNICAL FIELD

Described herein is the combination of immune agonists targeting immune cells (e.g., nanoparticles delivering STING and TLR4 agonists) with a combination of a MEK inhibitor, e.g., trametinib, and a CDK4/6 inhibitor, e.g., palbociclib, (referred to herein as T/P) treatment targeting tumor cells as an immunotherapy for cancer, e.g., PDAC. The combination of these treatments leads to (a) enhanced NP uptake in multiple cell types in the TME, (b) increased antigen presentation on tumors cells as well as APCs, and (c) CD8+ T cell mediated long-term tumor control that is interferon signaling dependent.

### BACKGROUND

Pancreatic ductal adenocarcinoma (PDAC) is a devastating disease with a dismal 5-year survival rate of 13% (1), largely due to the physiological makeup of the TME that promotes tumor advancement and limits effective treatment options. Characterized by a hallmark desmoplastic stroma, poor vascularization, and immunosuppression, the PDAC TME hinders effective drug delivery, drives chemo-resistance, and blocks the activation and infiltration of cytotoxic immune cells (2). Though immune checkpoint blockade (ICB) therapies targeting inhibitory checkpoints such as PD-1 and CTLA-4 on T cells have demonstrated durable responses in some cancer types, they have not shown efficacy in the immune suppressed PDAC TME that is devoid of CD8+ cytotoxic T lymphocytes (CTLs) and Natural Killer (NK) cells as well as antigen-presenting cells (APCs) such as dendritic cells (DCs) necessary to sustain anti-tumor T cell immunity (3-6). In addition, pancreatic tumor cells themselves have poor immunogenicity and antigenicity mediated in part by suppression of interferon signaling by oncogenic KRAS driver mutations (7).

### SUMMARY

Pancreatic ductal adenocarcinoma has quickly risen to become the 3rd leading cause of cancer-related death. This is in part due to its fibrotic tumor microenvironment (TME) that contributes to poor vascularization and immune infiltration and subsequent chemo-and immunotherapy failure. Here we investigated an innovative immunotherapy approach combining delivery of STING and TLR4 innate immune agonists via lipid-based nanoparticle (NP) co-encapsulation with senescence-inducing RAS-targeted therapies that can remodel the immune suppressive PDAC TME through the senescence-associated secretory phenotype. Treatment of transplanted and autochthonous PDAC mouse models with these regimens led to enhanced uptake of NPs by multiple cell types in the PDAC TME, induction of type I interferon and other pro-inflammatory signaling, increased antigen presentation by tumor cells and antigen presenting cells, and subsequent activation of both innate and adaptive immune responses. This two-pronged approach produced potent T cell-driven and Type I interferon-mediated tumor regressions and long-term survival in preclinical PDAC models dependent on both tumor and host STING activation. STING and TLR4-mediated Type I interferon signaling were also associated with enhanced NK and CD8⁺ T cell immunity in human PDAC. Thus, combining localized immune agonist delivery with systemic tumor-targeted therapy can synergize to orchestrate a coordinated Type I interferon-driven innate and adaptive immune assault to overcome immune suppression and activate durable anti-tumor T cell responses against PDAC.

As shown herein, the combination of STING/TLR4 agonist nanoparticles (NPs) and a combination of the MEK inhibitor trametinib and CDK4/6 inhibitor palbociclib (T/P) T/P treatment synergizes to (a) lead to enhanced NP uptake in multiple cell types in the TME, (b) increased antigen presentation on tumors cells as well as APCs, and (c) CD8+ T cell mediated long-term tumor control that is interferon signaling dependent. In some embodiments, the methods are used to treat cancer, e.g., KRAS mutant cancers, e.g., KRAS mutant PDAC or lung cancer.

Provided herein are compositions comprising (i) STING/TLR4 agonist nanoparticles (NPs), (ii) a MEK inhibitor, and (iii) a CDK4/6 inhibitor, in a pharmaceutically acceptable carrier.

Additionally provided herein are methods of treating cancer in a subject. The methods comprise administering a therapeutically effective amount of (i) STING/TLR4 agonist nanoparticles (NPs) and (ii) a combination of a MEK inhibitor and a CDK4/6 inhibitor.

Further, provided herein are compositions as described herein, e.g., comprising STING/TLR4 agonist nanoparticles (NPs) and a combination of a MEK inhibitor and CDK4/6 inhibitor, for use in a method of treating cancer in a subject.

In some embodiments, the MEK inhibitor is trametinib, and the CDK4/6 inhibitor is palbociclib, and/or in some embodiments, the STING/TLR4 agonists comprise cyclic-diguanosine monophosphate (cdGMP) and monophosphoryl-lipid A (MPLA). In some embodiments, the NPs comprise DOPC (1,2-dioleoyl-sn-glycero-3-phophocholine), DSPC (1,2-distearoyl-sn-glycero-3-phosphocholine), cholesterol, and a PEG-conjugated lipid, and optionally DOPG (1,2-dioleoyl-sn-glycero-3-phospho-(1′-rac-glycerol)). In some embodiments, the PEG-conjugated lipid is PEG-DSPE, optionally mPEG 2000-DSPE (methoxy-poly(ethyleneglycol)-2000 1,2-distearoyl-sn-glycero-3-phophoethanolamine-N) or amine-PEG2000-DSPE (amine-poly(ethylene glycol)-2000 1,2-distearoyl-sn-glycero-3-phophoethanolamine-N). As described herein, the PEG-containing compounds can include other PEGs in addition to or as an alternative to PEG2000, e.g., PEG1000, PEG3500, PEG5000, or PEG10000. In some embodiments, the NPs further comprise a targeting moiety on the surface of the NPs, optionally comprising an ECM peptide or an EGFR peptide.

In some embodiments, the cancer is lung cancer or pancreatic ductal adenocarcinoma (PDAC), optionally KRAS mutant lung cancer or PDAC.

In some embodiments, the MEK inhibitor and CDK4/6 inhibitor are administered before the STING/TLR4 agonist nanoparticles (NPs), or concurrently with the STING/TLR4 agonist nanoparticles (NPs).

In some embodiments, the methods comprise administering a composition as described herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### DESCRIPTION OF DRAWINGS

FIGS. 1A-J. Systemic administration of NPs can deliver cargo to tumor and immune cells in PDAC TME with minimal toxicity. (A) Schematic representation of immuno-NP design. (B) NP hydrodynamic size as assessed by dynamic light scattering (DLS). (C) Measurement of NP surface charge as assessed by zeta potential. (D) KPCI PDAC tumor cells expressing luciferase-GFP were injected orthotopically into the pancreas of 8-12 week old C57BL/6 female mice. Following tumor formation, mice received a single dose of fluorescently labeled immuno-NPs by intravenous (i.v.) injection. Flow cytometry analysis of DiI⁺ NP uptake in indicated cell types 48 hrs later is shown (n=7 mice per group). Tumor cells were defined as GFP⁺, and stromal cells as CD45-GFP. (E) PDAC-bearing KPC GEMM mice were i.v. injected with a single dose of fluorescently labeled immuno-NPs. Flow cytometry analysis of DiD-labeled NP uptake in different cell types 48 hrs later is shown (n=3 mice per group). (F) Representative immunofluorescence (IF) staining of KPCI orthotopic transplant PDAC tumors for Dil-labeled immuno-NPs.

Scale bars, 100 μm. (G) Representative immunofluorescence (IF) staining of KPC GEMM PDAC tumors for DiD-labeled immuno-NPs. Scale bars, 100 μm. (H) Plasma ALT and AST levels in naïve wild-type (WT) C57BL/6 mice treated with empty-or immuno-NPs weekly and vehicle or trametinib (1 mg/kg) and palbociclib (100 mg/kg) 4 times per week for 3 weeks (n=3 to 5 mice per group). Dotted lines indicate established range for normal AST and ALT levels. (I) Representative hematoxylin and eosin (H&E) staining of livers from WT C57BL/6 mice treated as in (H). Scale bars, 100 μm. (J) Change in weight of naïve WT C57BL/6 mice treated as indicated by arrows (n=3 to 5 mice per group). Error bars, mean±SEM. P values were calculated using 2-way ANOVA with Tukey's post-tests. n.s., not significant.

FIGS. 2A-H. T/P pre-treatment enhances immuno-NP uptake, IFN and cytokine production, and antigen presentation in tumor cells and APCs. (A) Schematic of KPC orthotopic transplant model and 2-week treatment schedule. (B) Flow cytometry analysis of DiI-labeled NP uptake in indicated cellular compartments in KPC1 transplant PDAC tumors from mice treated with vehicle or trametinib (1 mg/kg) and palbociclib (100 mg/kg) for 2 weeks and empty- or immuno-NPs for 48 hrs (n=4 to 8 mice per group). Tumor cells were defined as GFP⁺, and stromal cells as CD45⁻GFP⁻. (C) RT-qPCR analysis of IFN pathway and SASP gene expression in KPC1 transplant PDAC tumors from mice treated as in (B) (n=3 mice per group). A.U., arbitrary units. (D) Representative IF staining of KPC GEMM PDAC tumors from mice treated as in (B) for IFNβ in DCs (CD11c⁺), macrophages (F4/80⁺), and tumor cells (CK19⁺). Quantification of mean fluorescent intensity (MFI) of total IFNβ levels in tissues is shown in last panel on right (n=3 mice per group). Scale bars, 100 μm. (E) Schematic of KPC1 cell line in vitro treatment schedule. (F) RT-qPCR analysis of IFN pathway and SASP gene expression in KPC1 PDAC cells treated with vehicle or trametinib (25 nM) and palbociclib (500 nM) for 1 week and empty-or immuno-NPs for 48 hrs (n=3 samples per group). A.U., arbitrary units. (G) Representative histograms (left) and quantification of MHC-I (H-2k^{b}) MFI (right) on KPCI PDAC cells treated as in (F) (n=6 samples per group). (H) RT-qPCR analysis of antigen presentation/processing gene expression in KPCI transplant PDAC tumors from mice treated as in (B) (n=3 mice per group). A.U., arbitrary units. Error bars, mean±SEM. P values were calculated using two-tailed, unpaired Student's t-test. ****P<0.0001, ***P<0.001, **P<0.01, *P<0.05. n.s., not significant.

FIGS. 3A-H. Combinatorial immuno-NP and T/P treatment activates NK and CD8⁺ T cell immunity in PDAC. (A to C) Flow cytometry analysis of total CD45⁺ immune cells (A), T cell numbers and activation markers (B), and NK cell numbers and activation markers (C) in KPC1 orthotopic transplant PDAC tumors from mice treated with vehicle or trametinib (1 mg/kg) and palbociclib (100 mg/kg) for 2 weeks and empty-or immuno-NPs for 48 hrs (n=6 to 8 mice per group). (D) Immunohistochemical (IHC) staining of KPC1 orthotopic transplant PDAC tumors from mice treated as in (A). Quantification of the number of degranulating Granzyme B (GZMB)+cells per field is shown inset (n=3 to 6 mice per group). Scale bar, 50 μm. (E) IF staining of PDAC tumors from KPC GEMM mice treated as in (A) (left). Quantification of NK1.1⁺ NK cell and CD⁸⁺ T cell MFI is shown on right (n=3 mice per group). Scale bars, 100 μm. (F) IF staining for TNF≢0 in CD11c⁺ DCs (left) and F4/80⁺ macrophages (right) in PDAC tumors from KPC1 transplant mice treated as in (A). Scale bars, 100 μm. (G) Quantification of combined TNFα MFI in macrophages and DCs from IF staining in (F) (n=3 mice per group). (H) Flow cytometry analysis of DCs in KPC1 transplant PDAC tumors from mice treated as in (A) (n=6 to 8 mice per group). Error bars, mean±SEM. P values were calculated using two-tailed, unpaired Student's t-test. ****P<0.0001, ***P<0.001, **P<0.01, *P<0.05. n.s., not significant.

FIGS. 4A-F. Immuno-NP and T/P regimens produce tumor control and substantially increase overall survival in preclinical PDAC models. (A) Waterfall plot of the response of KPC1 transplant PDAC tumors after treatment with vehicle or trametinib (1 mg/kg) and palbociclib (100 mg/kg) 4 times per week and empty- or immuno-NPs weekly for 2 weeks (n=12 to 13 mice per group). (B) H&E staining of KPC1 transplant PDAC tumors from mice treated with vehicle or trametinib (1 mg/kg) and palbociclib (100 mg/kg) for 2 weeks and empty- or immuno-NPs for 48 hrs. Quantification of percent of tumor area covered in necrosis is shown inset (n=5 to 6 mice per group). Scale bar, 500 μm. (C) Kaplan-Meier survival curve of mice harboring KPC1 transplant PDAC tumors treated with vehicle or trametinib (1 mg/kg) and palbociclib (100 mg/kg) 4 times per week and emptyor immuno-NPs weekly (n=7 to 8 mice per group). (D) Waterfall plot of the response of KPC GEMM PDAC tumors to treatment as in (A) (n=5 to 9 mice per group). (E) H&E staining of KPC GEMM PDAC tumors from mice treated as in (B). Quantification of percent of tumor area covered in necrosis is shown inset (n=4 to 7 mice per group). Scale bar, 500 μm. (F) Kaplan-Meier survival curve of PDAC-bearing KPC GEMM animals treated as in (C) (n=8 to 10 mice per group). Arrows indicate when mice were taken off treatment. Error bars, mean±SEM. P values were calculated using two-tailed, unpaired Student's t-test (A and D) or log-rank test (C and F). ****P<0.0001, ***P<0.001, **P<0.01, *P<0.05. n.s., not significant.

FIGS. 5A-J. Tumor and host STING signaling contribute to anti-tumor immune responses to therapy. (A) Schematic of orthotopic transplantation of sg. Scramble or sg. Sting KPCI cells into WT or Sting^{-/-} mice and treatment. (B) Immunoblots of control or sg. Sting KPC1 PDAC cells. (C) Representative IHC staining of sg. Scramble or sg. Sting KPC1 transplant PDAC tumors from WT or Sting^{-/-} mice treated with trametinib (1 mg/kg) and palbociclib (100 mg/kg) for 2 weeks and immuno-NPs for 48 hrs. Scale bar, 100 μm. (D) Representative H&E staining of sg. Scramble or sg. Sting KPCI transplant tumors from WT or Sting^{-/-} mice treated as in (C) (top). Scale bar, 500 μm. Bottom, quantification of percent of tumor area covered in necrosis (n=8 mice per group). (E to J) Flow cytometry analysis of total CD45⁺ immune cell (E), CD8⁺ T cell (F), NK cell (G), CD4⁺ T cell (H), B cell (I), and DC (J) numbers and activation markers in sg. Scramble or sg. Sting KPC1 transplant tumors from WT or Sting^{-/-} mice treated as in (C) (n=8 to 9 mice per group). Error bars, mean±SEM. P values were calculated using two-tailed, unpaired Student's t-test. ****P<0.0001, ***P<0.001, **P<0.01, *P<0.05. n.s., not significant.

FIGS. 6A-H. Immuno-NP and T/P therapy efficacy driven by IFNAR-dependent NK and CD8⁺ T cell immune surveillance. (A) Kaplan-Meier survival curve of mice harboring KPCI transplant PDAC tumors treated with trametinib (1 mg/kg) and palbociclib (100 mg/kg) 4 times per week, immuno-NPs weekly, and blocking antibodies against NK1.1 (PK136 ; 250 μg), CD8 (2.43; 200 μg), or IFNAR-1 (MAR15A3; 200 μg) twice per week (n=7 mice per group). (B) Waterfall plot of the response of KPC1 transplant PDAC tumors to 2 weeks of treatment as in (A) (n=4 to 7 mice per group). (C) H&E staining of KPCI transplant PDAC tumors from mice treated with trametinib (1 mg/kg), palbociclib (100 mg/kg), and IFNAR-1 (MAR15A3; 200 μg) blocking antibodies for 2 weeks and immuno-NPs for 48 hrs. Scale bar, 500 μm. Quantification of percent of tumor area covered in necrosis is shown inset (n=3 to 4 mice per group). (D to H) Flow cytometry analysis of CD8⁺ T cell (D), NK cell (E), CD4⁺ T cell (F), B cell (G), and DC (H) numbers and activation markers in KPCI transplant PDAC tumors from mice treated as in (C) (n=16 to 17 mice per group). Error bars, mean±SEM. P values were calculated using log-rank test (A) or two-tailed, unpaired Student's t-test (B to H). ****P<0.0001, ***P<0.001, **P<0.01, *P<0.05. n.s., not significant.

FIGS. 7A-B. STING and TLR4 expression and Type I interferon signaling correlate with NK and T cell immunity in human PDAC. (A to B) Pearson's correlation analysis plots comparing NK and T cell signatures with expression of STING (TMEM174), TLR4, and downstream interferon signaling pathway genes in human PDAC transcriptomic data from Bailey et al. (36) (A) and Moffitt et al. (37) (B) (n=91 to 145 samples). Pearson's correlation coefficient (R) values are displayed. P values were calculated using a two-tailed, unpaired Student's t-test.

FIG. 8. Systemically delivered immuno-NPs accumulate in PDAC tumors and liver but not normal pancreas. (A) Characterization of MPLA (2 hr) and cdGMP (2 and 4 hr) encapsulation stability in PBS.

FIGS. 9A-H. T/P treatment enhances immuno-NP deposition 69 into the PDAC TME without inducing systemic inflammatory toxicities. (A) Flow cytometry analysis of MFI of DiI-labeled NP uptake in indicated cellular compartments in KPC1 transplant PDAC tumors from mice treated with vehicle or trametinib (1 mg/kg) and palbociclib (100 mg/kg) for 2 weeks and empty-or immuno-NPs for 48 hrs (n=4 to 8 mice per group). Tumor cells were defined as GFP+, and stromal cells as CD45−GFP−. (B) Biodistribution analysis following Spectrum imaging of fluorescent NPs in whole organs and blood plasma ex vivo harvested from KPC1 transplant mice 48 hr after treatment as in (A) (n=7 to 8 mice per group). Data in dotted inset on right are biodistribution measurements for tumors only. (C). Representative tissue cytometry image of a KPC GEMM pancreas from a mouse treated as in (A) stained for CK19+ tumor cells and NPs by immunofluorescence (IF). CK19 was used to distinguish tumor (R1) from adjacent normal (R2) regions. Quantification of NP signal MFI in normal and tumor regions is shown on right (n=3 mice per group, 1-3 images per mouse pancreas). (D) Organ masses in KPCI transplant mice treated as in (A) (n=7 to 8 mice per group). (E) Representative hematoxylin and eosin (H&E) staining of livers from KPC GEMM mice treated as in (A). Scale bars, 100 μm. (F-G) Quantification of IFNβ and TNFα MFI in livers (F) and spleens (G) of KPC1 transplant mice treated as in (A) as assessed by immunofluorescence staining (n=3 mice per group). (H) Multiplexed cytokine array analysis of blood from naive WT C57BL/6 mice treated with empty or immuno-NPs weekly and vehicle or trametinib (1 mg/kg) and palbociclib (100 mg/kg) 4 times per week for 3 weeks (n=3 to 5 mice per group). Error bars, mean±SEM. P values were calculated using two-tailed, unpaired Student's t-test (A and B), 2-way ANOVA with Tukey's post tests (B, C, D, H), or ordinary 1-way ANOVA with Tukey's post-tests (F, G). ***P<0.0001, **P<0.001, **P<0.01, *P<0.05. n.s., not significant.

FIG. 10. Kaplan-Meier survival curve of mice harboring KPCI transplant PDAC tumors treated with empty-or immuno-NPs weekly, trametinib (1 mg/kg) and palbociclib (100 mg/kg) 4 times per week, and/or blocking antibodies against PD-1 (RMP 1.14; 200 μg) 3 times per week (n=6 to 8 mice per group). Error bars, mean±SEM. P values were calculated using two-tailed, unpaired Student's t-test (A to E) or log-rank test (F). ***P<0.001, **P<0.01, not significant.

### DETAILED DESCRIPTION

RAS-targeted therapies not only increase antigen presentation through upregulation of major histocompatibility complex (MHC) Class I (MHC-I) molecules on tumor cells, but can also induce cellular senescence and a subsequent senescence-associated secretory phenotype (SASP) including angiogenic and inflammatory factors that can remodel immune suppressive TMEs in dynamic ways (8-14). In KRAS mutant lung adenocarcinoma (LUAD) models, treatment with combinations of the MEK inhibitor trametinib (T) and CDK4/6 inhibitor palbociclib (P) that target downstream KRAS signaling induce a pro-inflammatory SASP leading to NK cell-mediated lung tumor regressions (12). In contrast, treatment with the same T/P regimens in KRAS mutant PDAC models induced a pro-angiogenic SASP that promoted vascular remodeling and endothelial activation leading to increased chemotherapy delivery, CTL trafficking, and anti-PD-1 ICB efficacy (13). These organ-specific differences in immune responses appear to be dictated by the resident microenvironment. In particular, myofibroblasts prevalent in the PDAC TME contribute to suppression of pro-inflammatory SASP factors and effective NK and CD8⁺ T cell immunity following T/P treatment (15). Notably, targeting the mechanisms of TME-driven immune suppression led to reactivation of interferon regulatory factor (IRF) expression and downstream interferon signaling that are normally induced in LUAD but repressed in PDAC, suggesting that approaches to engage IFN signaling could be a means to activate CTL activity in the PDAC.

The pancreatic TME presents multiple immune suppressive hurdles that must be overcome for effective immunotherapy, including (a) a desmoplastic stroma contributing to physical exclusion and chemical inhibition of immune cells, (b) a poorly vascularized matrix leading to poor delivery of drugs and infiltration of peripheral immune cells, (c) a lack of cytotoxic NK and T lymphocytes that drive tumor eradication, (d) suppressive myeloid populations that inhibit lymphocyte activation, (e) few DCs to present antigen to T cells, and (f) low neo-antigen loads and dysfunctional antigen presentation circuitry in tumor cells allowing them to escape immune detection (2, 5). Though other groups have started to show promising early clinical results with neo-antigen vaccines (38), myeloid reprogramming (39, 40), and stromal remodeling agents (41, 42) that target some of these immune suppressive mechanisms, none of these therapies have yet to be clinically approved.

The Stimulator of Interferon Genes (STING) pathway is a major regulator of type I interferon production and has emerged as an important innate immune pathway that can enhance anti-tumor NK and T cell immunity (16). Upon binding 2′-3′-cyclic-GMP-AMP (cGAMP), a second messenger produced by cyclic GMP-AMP synthase (cGAS) following recognition of cytoplasmic double-stranded DNA, STING stimulates downstream activation of IRF3 and NF-κB transcriptional activity to drive type I interferons (e.g. IFNβ) and other pro-inflammatory cytokines and chemokines, including those associated with the SASP (17-20). Administration of cGAMP and other synthetic STING agonists have been shown to stimulate antigen-presenting DCs, reprogram immune suppressive macrophages, reduce inhibitory regulatory T cell (Treg) numbers, and increase CD8⁺ T cell activation, leading to anti-tumor effects in preclinical PDAC models (21-23). Though promising, the clinical development of STING agonists as potential immunotherapies has been constrained by (a) their unfavorable pharmacokinetics and poor bioavailability due to limited cellular uptake and half-life in circulation, (b) adverse toxicities and immune suppressive effects associated with systemic administration, and (c) the inaccessibility of some tumor sites, including the pancreas, to intratumoral administration (24-26).

To overcome these limitations, we have designed lipid-based nanoparticles (NPs) that allow for systemic delivery of payloads of STING agonists that are preferentially deposited and taken up by APCs within the “leaky” perivascular region of tumors because of their “stealth” surface coating and small size (27). Taking advantage of the fact that nanoparticles can deliver multiple immune-stimulating agonists as cargos, NPs were loaded with not only the STING agonist cyclic di-guanosine monophosphate (cdGMP), but also the Toll-like receptor 4 (TLR4) agonist monophosphoryl lipid A (MPLA) that can additionally stimulate Type I interferon responses (28, 29). We have shown in melanoma and triple-negative breast cancer models that systemic co-delivery of STING and TLR4 agonists in NPs (hereafter immuno-NPs) drives their access to and uptake into the TME of even poorly vascularized tumors and leads to synergistic and robust IFNβ production as compared to NPs carrying either single agonist alone (27, 30-32). Immuno-NP administration in these models resulted in potent activation of innate (APCs, NK cells) and adaptive (CTL) immune responses, reduced tumor growth, and prolonged survival that could not be achieved with free agonist delivery or even anti-PD1 ICB (27, 30-32).

We hypothesized that combining T/P and immuno-NP therapy would orchestrate a coordinated remodeling of immune suppressive networks within immune cells, tumor cells, and the vasculature in the PDAC TME to produce durable anti-tumor T cell responses. Here, using syngeneic transplant and autochthonous PDAC mouse models, we found that combined treatment led to enhanced immuno-NP uptake in multiple cell types in the TME, synergistic activation of both type I interferons and SASP-associated cytokines and chemokines, and upregulated antigen presentation on tumor cells and APCs that culminated in sustained interferon alpha and beta receptor subunit 1 (IFNAR)-dependent and CD8⁺ T cell-mediated anti-tumor immune responses against PDAC.

Thus, described herein is a multi-pronged approach combining innate immune agonist and tumor-targeting senescence-inducing therapies to target many of the immune suppressive mechanisms in PDAC simultaneously. There was remarkable synergy between T/P and immuno-NP therapies that could not be predicted from each monotherapy, including increased NP delivery and uptake of immune agonists in the PDAC TME, enhanced Type I interferon and cytokine signaling and antigen presentation in both tumor cells and DCs, reduced numbers of suppressive myeloid, Treg, and B cell populations, and potent NK and CD8⁺ T cell activation that culminated in even some complete tumor responses in preclinical animal models. Mechanistically, Type I interferons produced by STING activation in both tumor and host cells and their signaling through IFNAR⁺ immune cells were key to these therapeutic responses, implying that reactivating inflammatory signaling and antigen presentation pathways in tumor as well as immune cells may be pivotal in overcoming the multiple mechanisms of immune suppression in PDAC and achieving immune-mediated tumor control.

STING agonists have been actively pursued as immune oncology agents in PDAC as well as other solid tumor malignancies as a means to activate Type I interferon signaling that is critical for antigen presentation and productive anti-tumor innate and adaptive immune responses (16, 21-23). However, to date STING agonists have yet to show effective clinical utility in cancer in part through their limited cellular uptake, inflammatory toxicities associated with systemic administration, and long-term effects on T cell viability and exhaustion (24-26). A major innovation of this study is the ability to deliver STING agonists to diverse cell types within the hard-to-penetrate PDAC TME, through the design of lipid nanoparticles (NPs) engineered to preferentially deposit in the “leaky” tumor endothelium. Moreover, this nanomaterials-based drug delivery approach enables the effective and safe co-delivery of physically and chemically distinct STING (cdGMP) and TLR4 (MPLA) agonists, which can together drive robust downstream IRF3 and subsequent Type I interferon signaling (27, 30, 31, 43), without systemic toxicities. Remarkably, T/P pre-treatment, possibly through the vascular remodeling capabilities of its pro-angiogenic SASP, further increased the deposition of NPs in the PDAC TME. As such, this engineering approach augmented through senescence-induced vascular remodeling can overcome some of the drug delivery challenges that have been a major limitation to the effective treatment of PDAC and clinical development of STING agonists.

Unexpectedly, immuno-NPs were not only taken up by APCs in the perivascular regions of the PDAC TME, but also by tumor cells themselves, where they synergized with T/P-induced senescence to enhance the pro-inflammatory SASP in a tumor cell autonomous manner. The SASP can be a double-edged sword in cancer, with some SASPs promoting anti-tumor immune surveillance, while others pro-tumor immune suppression, and its context-dependent regulation is only beginning to be understood (14, 44). Indeed, whereas T/P-induced senescence drives immune-mediated tumor regressions in KRAS mutant lung cancers, it does not produce the same immune responses or tumor control KRAS mutant PDAC (12, 13, 15). Demonstrated herein is a new means of SASP regulation by which STING/TLR4-mediated signaling enhances not only IFNβ production but also a slew of pro-inflammatory SASP cytokines and chemokines that are normally repressed in the PDAC tumors even after T/P treatment to achieve NK and CD8⁺ T cell immune control. Though Type I interferon signaling through its receptor IFNAR is critical for cytotoxic lymphocyte immunity with this combination therapy, it is possible that other SASP-associated chemokines and cytokines that are also synergistically enhanced and that were previously implicated in activating NK and T cell immunity, including CCL2, CXCL9/10, and IL-12/-15/-18, could also contribute to immune responses to therapy. In addition, enhanced levels of MHC-I on tumor cells and MHC-II on DCs, presumably induced in response to interferon signaling, could also contribute to enhanced CD8⁺ T cell cytotoxicity following treatment, and if so may suggest rational combinations with neo-antigen or DC vaccines to sustain durable T cell responses against PDAC.

Immuno-NP and T/P treatment in human PDAC cells and analyses of patient PDAC samples suggests that these therapies can activate potent Type I interferon signaling, and that this is associated with enhanced NK and T cell immunity in human PDAC, highlighting the translational potential of our approach. Given differences in STING protein structure between mice and humans (18), our modular lipid-based NP approach can be adapted to substitute other STING agonists (e.g., cGAMP, diABZI) that may be more suitable for human use.

The results herein suggest that immuno-NP and T/P treatment may effectively synergize with anti-PD-1 ICB, as T cells become dysfunctional and express exhaustion checkpoints such as PD-1 following prolonged treatment. SASP activation produces vascular remodeling leading to increased uptake of chemotherapies and infiltration of T cells and synergy with anti-PD-1 immune checkpoint blockade (13) (U.S. Ser. No. 17/258,054, now U.S. Pat. No. 11/633,401). Given the recent development and promising responses observed with mutant allele-specific KRAS inhibitors in preclinical animal models and early human clinical trials of pancreatic cancer (45, 46), in the future it will be important to test these agents in combination with immune-stimulatory NPs as a means to further enhance on-target efficacy while minimizing off-target toxicity that has limited the clinical utility of MEK inhibitors as single agents (47).

The engineering approaches described herein, which target multiple cell types and immune suppressive barriers through induction of Type I interferon signaling in the PDAC TME, pave the way for coordinated innate and adaptive immune responses to achieve immunotherapy success that has thus far been elusive for PDAC patients. Thus, described herein are methods and compositions comprising combinations of STING/TLR4 agonist lipid nanoparticles (NPs) with a MEK inhibitor and a CDK4/6 inhibitor.

### Agonist Nanoparticles

The present methods and compositions can include immune agonist NPs, optionally comprising PEG, e.g., mPEG; cyclic-diguanosine monophosphate (cdGMP); and monophosphoryl-lipid A (MPLA). As shown herein, these NPs were designed to home to leaky tumor vasculature and can successfully deliver cargo containing immune agonists to perivascular space. Combining with T/P treatment further enhances their uptake into multiple cell types in the TME. The combination of T/P and agonist NPs leads to increased MHCII expression on APCs and MHC I expression on tumor cells, leading to enhanced antigen presentation. The combination of T/P and agonist NPs lead to sustained CD8+ T cell activation and T cell-mediated tumor regressions and long-term survival in preclinical mouse models of PDAC.

Stimulator of interferon response cGAMP interactor 1 (STING) agonists can include 5,6-dimethylxanthenone-4-acetic acid (DMXAA), ADU-S100, E7766, GSK3745417, cyclic-diguanosine monophosphate (cdGMP), cyclic dimeric adenosine monophosphate (c-dAMP), and cyclic guanosine monophosphate adenosine monophosphate (cGAMP), acridanone (e.g. 10-carboxymethyl-9-acridanone), 2-phenyl-2-thio-acetamide (e.g., C11), xanthone (e.g. α-Mangostin), amidobenzimidazole (e.g., diABZIs), 3-oxo-3,4-dihydro-2H-benzo[b][1,4]thiazine-6-carboxamide skeleton (e.g., G10), picolinamide skeleton (e.g. SINCRO), tetrahydro-dispiro-pyrazine-indene-hexaone (e.g., DSDP) and N-naphthalen-benzo dioxole carboxamide (e.g., BNBC). See, e.g., Huang et al., 2023.

TLR 4 agonists can include monophosphoryl-lipid A (MPLA), glycopyranosyl lipid A (GLA), GSK1795091, FP11, FP18, FP20-24, α-FP20, and FP200 (Romerio et al., J. Med. Chem. 2023, 66, 4, 3010-3029). Neoseptin-3 (Wang et al., 2016. 113(7 ) E884-E893).

Lipid nanoparticles for drug delivery that can be used in the present methods and compositions are known in the art. Exemplary lipid nanoparticles include DOPC (1,2-dioleoyl-sn-glycero-3-phophocholine), DSPC (1,2-distearoyl-sn-glycero-3-phosphocholine), cholesterol, a PEG-conjugated lipid, e.g., PEG-DSPE, e.g., amine-PEG2000-DSPE (amine-poly(ethylene glycol)- 2000 1,2-distearoyl-sn-glycero-3-phophoethanolamine-N) or mPEG2000-DSPE (methoxy-poly(ethyleneglycol)-2000 1,2-distearoyl-sn-glycero-3-phophoethanolamine-N), and optionally DOPG (1,2-dioleoyl-sn-glycero-3-phospho-(1′-rac-glycerol)). Other formulations can also be used; see, e.g., Kocabas et al., J. Contr. Release, 328(2020 ), pp. 587-595; Li et al., Chin. Chem. Lett., 32 (5) (2021), pp. 1615-1625; Medhi et al., ACS Appl. Nano Mater., 3(9 ) (2020), pp. 8557-8580; and Huang et al., Mater Today Bio. 2023 October 21:23: 100839. These can be used to encapsulate STING agonists and Toll-like receptor (TLR) agonists, e.g., TLR4 agonists.

See also Karathanasis et al., “Nanoparticle constructs for systemic co-delivery of anti-tumor agents”. U.S. patent application Ser. No. 16/989,473, filed Aug. 10, 2020; Chibaya et al., bioRxiv 2022.06.21.495523; Ruscetti M, Morris, JPM IV, Lowe SW. Combination Therapy with MEK Inhibitor and CDK4/6 Inhibitor to Treat Pancreatic Cancer, U.S. patent application Ser. No. 17/258,054, filed Jul. 8, 2021. Chibaya, L and Ruscetti, M. EZH2 inhibition in pancreatic cancer, PCT/US22/45163, filed Sep. 29, 2022, and Chibaya, L., Murphy, K.C., DeMarco, K.D. et al. EZH2 inhibition remodels the inflammatory senescence-associated secretory phenotype to potentiate pancreatic cancer immune surveillance. Nat Cancer (2023). doi. org/10.1038/s43018-023-00553-8, all of which are incorporated herein by reference.

### Targeting NPs

Given the diverse cell types targeted by these therapies, though systemic administration of NPs is able to safely and effectively deliver immune agonist cargo to the PDAC tumor site, NPs still deposit in the liver to some extent. In light of the tunability of the nanomaterials engineering approaches described herein, targeting moieties can be added to the NPs to better direct them to specific cell types in the PDAC TME, the PEG coating can be modulated to minimize uptake and retention in the liver (for examples, the PEG2000 compounds exemplified herein could be replaced with, or could also include PEG1000, PEG3500, PEG5000, or PEG10000, e.g., in alternating lengths achieved by synthesizing the particles with desired ratios; see, e.g., Zhou et al., ACS Nano 2018, 12, 10, 10130-10141; Kaminskas et al., Mol. Pharmaceutics 2008, 5, 3, 449-463; Gref et al., Adv Drug Deliv Rev. 1995 September; 16(2-3): 215-233; Duan and Li, Small. 2013 May 27; 9(9-10): 1521-32; Almeida et al., Nanomedicine (Lond). 2011 July; 6(5): 815-35; Suk et al., Adv Drug Deliv Rev. 2016 Apr. 1; 99(Pt A): 28-51), and the T/P can be encapsulated into the NPs to further optimize their tumor delivery and on-target effects.

Targeting moieties can be present on the surface of the NPs, e.g., covalently linked to the PEG chains on the surface of NPs, and can include ECM peptide (amino acid sequence: CREKA) or EGFR peptide (amino acid sequence: CYHWYGYTPQNVI).

### MEK Inhibitors and CDK4 Inhibitors

The present methods and compositions can include MEK inhibitors and CDK4inhibitors. Exemplary MEK inhibitors include trametinib, cobimetinib, binimetinib, selumetinib, PD-325901, TAK-733, CI-1040 (PD184352), PD0325901, MEK162, AZD8330, GDC-0623, refametinib, pimasertib, RO4987655, RO5126766, WX-554, HL-085, CInQ-03, G-573, PD184161, PD318088, PD98059, RO5068760, U0126, and SL327. Exemplary CDK4/6 inhibitors include palbociclib, ribociclib, and abemaciclib. Preferably, a combination of trametinib and palbociclib is used.

In some embodiments, the T/P are encapsulated in NPs for delivery. See also Ruscetti M, Morris, JPM IV, Lowe SW. Combination Therapy with MEK Inhibitor and CDK4/6 Inhibitor to Treat Pancreatic Cancer, U.S. patent application Ser. No. 17/258,054, filed Jul. 8, 2021. Chibaya, L and Ruscetti, M. EZH2 inhibition in pancreatic cancer, PCT/US 22/45163, filed Sep. 29, 2022. Karathanasis et al., “Nanoparticle constructs for systemic co-delivery of anti-tumor agents”. U.S. patent application Ser. No. 16/989,473, filed Aug. 10, 2020; Chibaya et al., bioRxiv 2022.06.21.495523; and Chibaya, L., Murphy, K.C., DeMarco, K.D. et al. EZH2 inhibition remodels the inflammatory senescence-associated secretory phenotype to potentiate pancreatic cancer immune surveillance. Nat Cancer (2023).

doi. org/10.1038/s43018-023-00553-8, all of which are incorporated herein by reference.

In some embodiments, the compositions comprise LNPs encapsulating the STING and TLR4 agonists, and the MEK and CDK4/6 inhibitors are in solution, either in the same or a different solution; alternatively, one or both of the MEK and CDK4/6 inhibitors can also be encapsulated in NPs, either in the same NPs as the STING and TLR4 agonists, or in separate NPs.

### Methods of Treatment

The compositions described herein can be used to treat subjects with cancer, e.g., any KRAS mutant cancer, for example KRAS mutant colon cancer, cholangiocarcinoma, endometrial cancer, PDAC, or lung cancer, e.g., KRAS mutant lung cancer. In some embodiments, the cancer is PDAC. The methods generally include administering a therapeutically effective amount of combinations of STING/TLR4 agonist lipid nanoparticles (LNPs) with a MEK inhibitor and a CDK 4/6 inhibitor, as described herein. In some embodiments, the agonist LNPs and the MEK and CDK4/6 inhibitors are administered together; alternatively and/or in addition, the MEK and CDK 4/6 inhibitors are administered before (e.g., for 1-2 weeks before) administration of the agonist LNPs, and/or before and during administration of the agonist LNPs. As noted above, the LNPs encapsulate the STING and TLR4 agonists, and the MEK and CDK4/6 inhibitors can be in solution, either in the same or a different solution; alternatively, one or both of the MEK and CDK4/6 inhibitors can also be encapsulated in NPs, either in the same NPs as the STING and TLR4 agonists, or in separate NPs.

The methods can include systemic administration of one or more doses of the agonist LNPs and the MEK and CDK4/6 inhibitors to treat the cancer in the subject. A treatment can result in a reduction in tumor size or growth rate. Administration of a therapeutically effective amount of a treatment described herein can result in a reduction in tumor size or decreased growth rate, a reduction in risk or frequency of reoccurrence, a delay in reoccurrence, a reduction in metastasis, increased survival, and/or decreased morbidity and mortality, inter alia.

The present methods can also include administering an immunotherapy comprising a checkpoint inhibitor, e.g., an inhibitor of PD-1 signaling, e.g., an antibody that binds to PD-1, CD40, or PD-L1, or an inhibitor of Tim3 or Lag3, e.g., an antibody that binds to Tim3 or Lag3, or an antibody that binds to CTLA-4, or an antibody that binds to T-cell immunoglobulin and ITIM domains (TIGIT). These could be administered concurrently with MEK/CDK 4/6 and immune-NPs or after 2 week treatment with them.

Exemplary anti-PD-1 antibodies that can be used in the methods described herein include those that bind to human PD-1; an exemplary PD-1 protein sequence is provided at NCBI Accession No. NP_005009.2. Exemplary antibodies are described in U.S. Pat. Nos. 8,008,449; 9,073,994; and US20110271358, including PF-06801591, AMP-224, BGB-A317, BI 754091, JS001, MEDI0680, PDR001, REGN2810, SHR-1210, TSR-042, pembrolizumab, nivolumab, avelumab, pidilizumab, and atezolizumab.

Exemplary anti-CD40 antibodies that can be used in the methods described herein include those that bind to human CD40; exemplary CD40 protein precursor sequences are provided at NCBI Accession No. NP_001241.1, NP_690593.1, NP_001309351.1, NP_001309350.1 and NP_001289682.1. Exemplary antibodies include those described in WO2002/088186; WO2007/124299; WO2011/123489; WO2012/149356; WO2012/111762; WO2014/070934; US20130011405; US20070148163; US20040120948; US20030165499; and U.S. Pat. No. 8,591,900, including dacetuzumab, lucatumumab, bleselumab, teneliximab, ADC-1013, CP-870,893, Chi Lob 7/4, HCD122, SGN-4, SEA-CD 40, BMS- 986004, and APX005M. In some embodiments, the anti-CD40 antibody is a CD40 agonist, and not a CD40 antagonist.

Exemplary CTLA-4 antibodies that can be used in the methods described herein include those that bind to human CTLA-4; exemplary CTLA-4 protein sequences are provided at NCBI Acc No. NP_005205.2. Exemplary antibodies include those described in Tarhini and Iqbal, Onco Targets Ther. 3:15-25 (2010); Storz, MAbs. 2016 Jan; 8(1): 10-26; US 2009025274; U.S. Pat. Nos. 7,605,238; 6,984,720; EP1212422; U.S. Pat. Nos. 5,811,097; 5,855,887; 6,051,227; 6,682,736; EP1141028; and U.S. Pat. No. 7,741,345; and include ipilimumab, Tremelimumab, and EPR1476.

Exemplary anti-PD-L1 antibodies that can be used in the methods described herein include those that bind to human PD-L1; exemplary PD-L1 protein sequences are provided at NCBI Accession No. NP 001254635.1, NP_001300958.1, and NP_054862.1. Exemplary antibodies are described in US20170058033; WO2016/061142A1; WO2016/007235A1; WO2014/195852A1; and WO2013/079174A1, including BMS-936559 (MDX-1105), FAZ053, KN035, Atezolizumab (Tecentriq, MPDL3280A), Avelumab (Bavencio), and Durvalumab (Imfinzi, MEDI-4736).

Exemplary anti-Tim3 (also known as hepatitis A virus cellular receptor 2 or HAVCR2) antibodies that can be used in the methods described herein include those that bind to human Tim3; exemplary Tim3 sequences are provided at NCBI Accession No. NP 116171.3. Exemplary antibodies are described in WO2016071448; U.S. Pat. No. 8,552,156; and US PGPub. Nos. 20180298097; 20180251549; 20180230431; 20180072804; 20180016336; 20170313783; 20170114135; 20160257758; 20160257749; 20150086574; and 20130022623, and include LY 3321367, DCB-8, MBG453 and TSR-022.

Exemplary anti-Lag3 antibodies that can be used in the methods described herein include those that bind to human Lag3; exemplary Lag3 sequences are provided at NCBI Accession No. NP_002277.4. Exemplary antibodies are described in Andrews et al., Immunol Rev. 2017 March; 276(1): 80-96; Antoni et al., Am Soc Clin Oncol Educ Book. 2016; 35: e450-8; US PGPub. Nos. 20180326054; 20180251767; 20180230431; 20170334995; 20170290914; 20170101472; 20170022273; 20160303124, and include BMS- 986016.

Exemplary anti-TIGIT antibodies that can be used in the methods described herein include those that bind to human TIGIT; an exemplary human TIGIT sequence is provided at NCBI Accession No. NP_776160.2. Exemplary antibodies include AB154; MK-7684; BMS-986207; ASP8374; Tiragolumab (MTIG7192A; RG6058); (Etigilimab (OMP-313M32)); 313R12. See, e.g., Harjunpää and Guillerey, Clin Exp Immunol. 2020 May; 200(2): 108-119; and US PGPub. Nos. 20200062859 and 20200040082.

### Pharmaceutical Compositions and Methods of Administration

The methods described herein include the use of pharmaceutical compositions comprising or consisting of agonist LNPs and/or MEK and/or CDK4/6 inhibitors as an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions, e.g., a checkpoint inhibitor as described herein.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

The pharmaceutical compositions can be included in a container, pack, or dispenser together with instructions for administration.

### EXAMPLES

The invention is further described in the following examples, which do not limit the scope of the invention described in the claims.

### MATERIALS AND METHODS

The following materials and methods were used in the Examples below.

### Study Design

Sample sizes were determined based on those reported in previous publications (12, 13, 15) and no statistical method was used to predetermine sample size. The indicated sample size (n) represents biological replicates. All experiments were repeated independently 2-3 times. All samples that met proper experimental conditions were included in the analysis. For in vivo experiments, mice were randomized based on tumor burden as assessed by ultrasound imaging to achieve equal tumor volume between experimental groups. For in vitro experiments sample allocation was performed randomly. Data collection and analysis were not performed in a blinded manner.

### Nanoparticle Synthesis and Characterization

Dual agonist immuno-NPs were synthesized by pulsed ultrasonication. Equimolar amounts of DOPC (33.5 mol % 1,2-dioleoyl-sn-glycero-3-phophocholine, Avanti) and DSPC (33.5 mol % 1,2-distearoyl-sn-glycero-3-phosphocholine, Avanti) as well as DOPG (20 mol % 1,2-dioleoyl-sn-glycero-3-phospho-(1′-rac-glycerol), Avanti) were prepared in lipid films, along with 10 mol % cholesterol and 3 mol % mPEG 2000-DSPE [methoxy-poly(ethyleneglycol)-2000 1,2-distearoyl-sn-glycero-3-phophoethanolamine-N, Laysan Bio]. MPLA (Sigma-Aldrich) and cdGMP (Invivogen) were added, along with a lipophilic fluorescent Di tracer (i.e. Dil, DiD) at 0.1 mol % for NP tracking experiments. Following rehydration in PBS, samples were ultrasonicated using intermittent pulsing. Formed immuno-NPs were harvested and then dialyzed against sterile PBS. Dynamic light scattering (DLS) and zeta potential measurements were used to assess immuno-NP hydrodynamic size and surface, respectively, using a Malvern Panalytical Zetasizer. Commercially available kits were used to quantify cdGMP (Lucerna Technologies) and MPLA (Sigma Aldrich) encapsulation. Empty NPs lacking MPLA and cdGMP were used as a vehicle control.

### Targeted Agonist-and Inhibitor-np Synthesis and Characterization

NPs were synthesized by pulsed ultrasonication. Equimolar amounts of DOPC (43.5 mol % 1,2-dioleoyl-sn-glycero-3-phophocholine, Avanti) and DSPC (43.5 mol % 1,2-distearoyl-sn-glycero-3-phosphocholine, Avanti) were prepared in lipid films, along with 10 mol % cholesterol and 3 mol % amine-PEG 2000-DSPE [amine-poly(ethylene glycol)-2000 1,2-distearoyl-sn-glycero-3-phophoethanolamine-N, Laysan Bio]. To stabilize the matrix, DOPG (20 mol % 1,2-dioleoyl-sn-glycero-3-phospho-(1′-rac-glycerol), Avanti) can also be added if needed.

### Extracellular Matrix (ECM)-Targeted Agonist-NPs

MPLA (Sigma-Aldrich) and cdGMP (Invivogen) were added, along with a lipophilic fluorescent Di tracer (i.e. Dil, DiD) at 0.1 mol % for NP tracking experiments. Following rehydration in PBS, samples were ultrasonicated using intermittent pulsing. Formed immuno-NPs were harvested and then dialyzed against sterile PBS. Dynamic light scattering (DLS) and zeta potential measurements were used to assess immuno-NP hydrodynamic size and surface, respectively, using a Malvern Panalytical Zetasizer. Commercially available kits were used to quantify cdGMP (Lucerna Technologies) and MPLA (Sigma Aldrich) encapsulation. Empty NPs lacking MPLA and cdGMP were used as a vehicle control. Agonist-NPs with terminal amine-PEG chains were then reacted with sulfo-SMCC crosslinker (Thermo Fisher) for 10 min and ECM peptide (amino acid sequence: CREKA) reconstituted in PBS was added to this reaction for 50 min with gentle shaking. Targeted agonist-NPs were dialyzed against PBS for 30 min and stored immediately at 4° C.

### EGFR-Targeted Inhibitor-NPs

NP synthesis, characterization, and targeting are performed identically to ECM-targeted agonist-NPs, albeit with the use of trametinib (MedChemExpress) and palbociclib (MedChemExpress) inhibitors in lieu of agonists and EGFR peptide (amino acid sequence: CYHWYGYTPQNVI) in lieu of CREKA peptide. Inhibitor encapsulation is characterization by UV absorption.

### Animal Studies

All mouse experiments in this study were approved by the University of Massachusetts Chan Medical School Internal Animal Care and Use Committee. Mice were maintained under specific pathogen-free conditions, and food and water were provided ad libitum. C57BL/6 mice were purchased from Charles River Laboratories and KPC GEMM mice were bred in-house. Tmem 173 (Sting)^{-/-} and Ifnar I^{-/-} mice on a C57BL/6 background were generously provided by Z. Jiang and F. Humphries, respectively, and bred in-house.

### Pancreas Orthotopic Transplant Models

5×10⁴ KPCI cells were resuspended in 25 μl of Matrigel (Matrigel, BD) diluted 1:1 with cold advanced DMEM/F 12 media and transplanted into the pancreas of 8-12-week-old C57BL/ 6 female mice, or 8-16-week-old Sting^{-/-} and Ifnar 1^{-/-} male and female mice. After administering anesthesia using 2-3% isoflurane, an incision was performed on the left side of the abdomen. Subsequently, the cell suspension was injected into the tail region of the pancreas using a Hamilton Syringe. The injection's success was confirmed by the presence of a fluid bubble without any indications of leakage into the abdominal cavity. The abdominal wall was closed using an absorbable Vicryl suture (Ethicon), and the skin was secured with wound clips (CellPoint Scientific Inc.). Mice were then monitored for tumor development using ultrasound imaging. One week after transplantation, mice were randomized into different treatment groups based on tumor volume. Following sacrifice, a portion of the pancreas tumor tissue was preserved in 10% formalin for fixation, while others were used for OCT frozen blocks and flow cytometry analysis.

### KPC Genetically Engineered Mouse Model (GEMM)

P48-Cre; Kras^{LSL-G12D/wt}; Trp53^{fl/wt} (KPC) GEMMs were generated by interbreeding P48-Cre, Kras^{LSL-G12D/wt}, and Trp53^{fl/fl} strains on a C57BL/6 background. Tumor development was monitored using ultrasound imaging. Once tumors reached approximately 50 mm³ in volume, the mice were enrolled and randomized into different treatment groups based on tumor volume. After sacrificing the mice, pancreatic tumor tissue was divided for single cell suspensions for flow cytometry, 10% formalin fixation for immunohistochemistry (IHC), and OCT frozen blocks for immunofluorescence (IF) analysis.

### Systemic Safety Studies

Naïve 10-16-week-old C57BL/ 6 mice were treated weekly with immuno-NPs containing 7 μg of cdGMP and MPLA each by intravenous (i.v.) injection alone or in combination with T/P administered 4 days on/3 days off per week for 3 consecutive weeks. Animal weight was recorded daily. Following 3 weeks of treatment, mice were euthanized and blood and major organs, including pancreas, liver, spleen, and lungs, were harvested and weighed. Spectrum imaging was performed on plasma and organs to quantify NP biodistribution. Blood and organs were then processed for flow cytometry analysis. Blood plasma samples were analyzed for ALT/AST levels by the UMass Chan Medical School Analytical Core and for proinflammatory cytokines using a multiplex immunoassay (Mouse Cytokine/Chemokine 44-Plex array) from Eve Technologies (Calgary, CANADA). Liver toxicity was assessed histopathologically using H&E-stained sections.

### Drug Treatments and Neutralizing Antibodies

Trametinib was dissolved in a solution containing 0.5% hydroxypropyl methylcellulose and 0.2% Tween-80 and palbociclib in 50 mM sodium lactate buffer (pH 4). PDAC-bearing mice were treated with vehicles or trametinib (1 mg/kg) and palbociclib (100 mg/kg) (LC Laboratories) orally for four consecutive days followed by three days without treatment for two weeks or until survival endpoint. For short-term 48 hr or 2-week treatment studies, PDAC-bearing mice received a single dose of control empty NPs or immuno-NPs carrying 7 μg of cdGMP and MPLA each by intravenous (i.v.) injection, and animals euthanized and tumors harvested 48 hrs after treatment. For long-term timepoint and survival experiments, PDAC-bearing mice received empty or immuno-NPs weekly. To deplete NK or CD8⁺ T cells, mice received intraperitoneal (i.p.) injections of an αNK1.1 (250 μg; PK136, BioXcell) or aCD8 (200 μg; 2.43, BioXcell) antibody twice per week, respectively. To neutralize IFNAR signaling, mice were i.p. injected with an αIFNAR-1 antibody (200 μg; MAR15A3, BioXcell) twice per week. For the immune checkpoint blockade (ICB) study, mice received i.p. injections of an αPD-1 (200 μg; RMP1-14, BioXcell) antibody three times per week. No toxicities (as assessed by changes in organ and animal weights and liver damage) were observed in animals treated with these compounds alone or in combination. Ultrasound imaging was performed every two weeks during the treatment period to monitor changes in PDAC tumor burden.

### Ultrasound Imaging

To stage and quantify tumor burden, high-contrast ultrasound imaging was performed using a Vevo 3100 System with a MS 250 13- to 24-MHz scanhead (VisualSonics). Tumor volume was analyzed using Vevo LAB software.

### Spectrum Imaging

To assess NP biodistribution, tumors, blood plasma, and major organs (liver, lungs, spleen, kidneys) were harvested, and NP fluorescence was imaged ex vivo using an IVIS-Spectrum CT system (Perkin-Elmer). Quantification was performed using Living Image software.

### Immunofluorescence (if)

Fresh tissues were embedded in OCT, frozen, and cut into 5 μm sections (taken from center of the tissue). Tissue sections were placed in humidity chambers for staining. In brief, samples were washed 3× with PBS prior to fixation with 2% PFA for 1 h at RT. PFA was removed and protein blocking solution (4% goat serum, 0.5% triton-X in PBS) was added for 20 min at RT. The following primary antibodies diluted in protein blocking solution were added to the tissue and incubated overnight at 4° C.: CK19(1:200; TROMA-III, Univ. of Iowa Developmental Studies Hybridoma Bank), IFNβ (1:100; polyclonal, Thermo Fisher), CD11c (1:100; N418, Thermo Fisher), F4/80 (1:100; A3-1, Thermo Fisher), TNFα (1:100; polyclonal, Thermo Fisher), p-IRF3 (1:100; polyclonal, Bioss Antibodies), CD8a (1:100; 53-6.7, Thermo Fisher), and NK1.1 (1:100; polyclonal, Thermo Fisher). Tissues were then washed 3× with PBS and secondary Alexa Fluor 405, 488, 568, or 647 dye-conjugated antibodies (Thermo Fisher) were added diluted 1:150 in protein blocking solution for 1 h at RT. Tissue sections were washed 3× with PBS and mounting media with or without DAPI (Vectashield) was added prior to applying a glass coverslip. High-magnification images were obtained using a Nikon Al confocal microscope or Leica Thunder Live Cell and 3D Assay Imager. Whole tissue images were obtained using a TissueFAXS SL widefield tissue cytometer. Fluorescence was analyzed and quantified using Fiji/ImageJ.

### Immunohistochemistry (ihc)

Tissues were fixed overnight in 10% formalin, embedded in paraffin, and cut into 5 μm sections. Hematoxylin and eosin (H&E) and immunohistochemical staining were performed using standard protocols. For immunohistochemistry, sections were deparaffinized, rehydrated with decreasing concentrations of ethanol in water, and boiled in a pressure cooker for 20 minutes in 10 mM citrate buffer (pH 6.0). Endogenous peroxidases were quenched by incubating the slides in 3% hydrogen peroxide for 20 min. The sections were then washed 2× with PBS and the following primary antibodies were incubated overnight at 4° C.: FOXP3 (1:100; FKJ- 16s, eBioscience), Granzyme B (GZMB) (1:100; AB4059, Abcam), and STING (1:200; D2P2F, Cell Signaling). HRP-conjugated secondary antibodies (Vectastain Elite ABC-HRP Kits: Rat, PK-6104; Rabbit, PK-6101) were applied for 30 minutes and visualized with DAB (Vector Laboratories; SK-4100).

For quantification of FOXP3⁺ Tregs and GZMB⁺ immune cells, 5-10 high power 20× fields per section were counted and averaged using ImageJ software.

Tumor necrosis was assessed by quantifying the percentage of total PDAC tumor area covered in necrotic tissue from H&E-stained sections using ImageJ software.

### Cell Lines and In Vitro Drug Treatments

PANC-1, Panc02, RAW 264.7, and 293T cells were obtained from the American Type Culture Collection (ATCC). The murine KPC1 PDAC cell line was generated as described previously (13). KPC1 cells were transduced with an MSCV-luciferase (luc)-IRES-GFP retroviral construct to visualize and track tumor cells following in vivo transplantation. Retroviruses were produced by co-transfecting Gag-Pol expressing 293T cells with the appropriate expression and envelope vectors (VSV-G). After transduction, the cells were purified by FACS sorting the GFP⁺ population using a FACSAria (BD Biosciences). All cells were cultured in a humidified incubator at 37° C. with 5% CO₂ and grown in DMEM supplemented with 10% FBS and 100 IU/ml penicillin/streptomycin (P/S). KPC1 cells were grown on culture dishes coated with 100 μg/ml collagen (PureCol) (5005; Advanced Biomatrix). All cell lines used tested negative for mycoplasma.Human cell lines were authenticated by their source repository.

Trametinib (S2673) and palbociclib (S1116) were purchased from Selleck Chemicals and MedChemExpress, dissolved in DMSO (vehicle) to obtain 10 mM stock solutions, and stored at −80° C. for in vitro studies. Nanoparticles were synthesized using identical methods to those used for in vivo studies as described above. Human and mouse cell lines were treated with 25 nM trametinib and 500 nM palbociclib for 7 days and received 1 dose of empty or immuno-NPs (carrying 7 μg each of cdGMP and MPLA) on day 5 prior to their harvesting 48 hrs later.

### Sting KO Cell Line Generation

Lentivirus was generated by transfecting 293T cells with psPAX 2, pMD 2.G, and LentiCRISPRv2 plasmids carrying an sgRNA against mouse Tmem173 (Sting) or a scrambled sequence using the Effectene reagent (Qiagen) following the manufacturer's instructions. pLentiCRISPRv2_mSTING_gRNA_2 and pLentiCRISPRv2_mSTING_scrambled_gRNA_2 were gifts from Nicolas Manel (Addgene plasmids #196626 and #196627). Following transduction of KPCI cells with lentivirus-containing media and polybrene (8 μg/ml), cell selection was performed with 4 μg/ml puromycin for 5 days. STING protein loss was confirmed by immunoblotting and immunohistochemistry.

### qRT-PCR

Total RNA was extracted from KPC cell lines or bulk PDAC tumor tissue using the RNeasy Mini Kit (Qiagen). Complementary DNA (cDNA) was obtained using TaqMan reverse transcription reagents (Applied Biosystems). Real time qPCR was performed in triplicate using Power SYBR™ Green PCR Master Mix (Applied Biosystems) on the StepOnePlus RealTime PCR System (Applied Biosciences). The comparative CT method (2^{-ΔΔCT}) was used to determine fold differences between the target gene and the reference gene GAPDH.

### Immunoblotting

Whole cell protein lysates were extracted using RIPA buffer (Cell signaling) supplemented with phosphatase inhibitors (5 mM sodium fluoride, 1 mM sodium orthovanadate, 1 mM sodium pyrophosphate, 1 mM β-glycerophosphate) and protease inhibitors (Protease Inhibitor Cocktail Tablets, Roche). Protein concentration was determined using a Bradford Protein Assay kit (Biorad). Proteins were separated by SDS-PAGE and transferred to polyvinyl difluoride (PVDF) membranes (Millipore) according to standard protocols. Membranes were blotted with antibodies (1:1,000) against phosphorylated (p)-STING^{S365} (D8F4W), STING (D2P2F), p-IRF3^{S396} (4D4G), IRF3 (D83B9), p-TBK1^{S172} (D52C2), TBK1 (E8I3G), p-p65^{S536} (93H1), and p65 (D14E12) from Cell Signaling in 5% milk in TBS blocking buffer. After primary antibody incubation, membranes were probed with an ECL anti-rabbit IgG secondary antibody (1:10,000) from GE Healthcare Life Science and imaged using Chemidoc Molecular Imaging System (BioRad). Protein loading was determined using a monoclonal β-actin antibody directly conjugated to horseradish peroxidase (1:20,000) from Sigma-Aldrich (A3854). Quantitation of western blot band intensity was done using Image J software.

### Flow Cytometry

To assess MHC-I surface protein levels on KPC cells cultured in vitro, drug-treated cells were trypsinized, resuspended in PBS supplemented with 2% FBS, and stained with an H-2k^{b} antibody (AF 6-88.5.5.3, eBioscience; 1:200) for 30 minutes on ice. Flow cytometry analysis was conducted using a BD LSR II instrument, and FlowJo software (TreeStar) was used for data analysis.

For in vivo sample preparation, pancreatic tumor tissues from tumor-bearing animals, as well as liver, lungs, spleen, and normal pancreas from naïve WT animals, were isolated and allocated for 10% formalin fixation, OCT frozen blocks, and/or flow cytometry analysis following treatment. To generate single cell suspensions for flow cytometry analysis, pancreas tumors were minced into small pieces with scissors, placed in 5 ml of collagenase buffer (1× HBSS with calcium and magnesium, 1 mg/ml Collagenase V, 0.1 mg/ml DNase I) in C tubes, and then processed using program 37C_m_TDK1_1 on a gentleMACS Octo dissociator with heaters (Miltenyi Biotec). For liver, lung, spleen, and normal pancreas, tissue harvests were diced into small 1 mm sections and then digested for 1 hr in 1 mg/mL Collagenase V prepared in serum-free DMEM medium at 37° C. The dissociated tissue was passed through a 70 μm cell strainer, centrifuged, and resuspended in PBS supplemented with 2% FBS. For blood samples, erythrocytes were removed following treatment with ACK lysing buffer to obtain leukocytes for staining. Samples were then incubated with the following antibodies for 30 minutes on ice: CD45 AF700 (30-F11; 1:320) for tumor or CD45 BV510 (30-F11; 1:200) for normal organs, NK1.1 BV605 or PE-Cy7 (PK136; 1:200), CD3 BV650(17A2; 1:300), CD8 PE-Cy7 (53 -6.7; 1:400), CD4 PE-Cy5 (GK1.5; 1:200), CD69 APC-Cy7 (H1.2F3; 1:200), F4/80 APC or APC-Cy7 (BM8; 1:200), CD11c BV785 (N418; 1:100) for tumors or CD11c PE (N418; 1:200) for normal organs, MHC-II (I-A/I-E) PE-Dazzle 594 (MS114.15.2; 1:200), Gr-1 (Ly-6 G/Ly-6C) Pacific Blue (RB6-8C5; 1:200), B220 (CD45R) PerCP-Cy5.5 (RA3-6B2; 1:400), CD44 BV786(IM7; 1:100), Sca-1 PerCP-Cy5.5 (D7; 1:100), PD-1 PE (CD279, RMP 1-30; 1:200) (Biolegend); and CD11b BUV395 (M1/70; 1:1,280) (BD Biosciences). DAPI was used to distinguish live/dead cells, and Dil and DiD fluorophores used to mark NPs. Flow cytometry was performed on an BD LSR II and FACSymphony A5, and CD45⁺ immune cell, CD45⁻ GFP⁺ tumor cell, CD45⁻GFP⁻ stromal cell, CD4⁺ and CD8⁺ CD3⁺ T cell, CD3⁻ NK1.1⁺ NK cell, CD3⁻ B220⁺ B cell, CD11b⁺F4/80⁺MHC-II⁺ macrophage, CD11b⁺Gr-1⁺ MDSC, CD11c⁺MHC-II⁺ dendritic cell numbers and levels of activation (CD69, CD44, Sca-1) and exhaustion (PD-1) markers and NP fluorophores (DiI/DiD) were analyzed using FlowJo (TreeStar).

To analyze Granzyme B (GZMB), IFNγ, and TNFα levels in NK and T cells, single cell suspensions from tumor tissue were resuspended in RPMI media supplemented with 10% FBS and 100 IU/ml P/S and incubated for 4 hours with PMA (20 ng/ml, Sigma-Aldrich), Ionomycin (1 μg/ml, STEMCELL technologies), and monensin (2 μM, Biolegend) in a humidified incubator at 37° C. with 5% CO₂. Cell surface staining was first performed with CD45 AF 700(30 -F11; 1:320), NK 1.1 BV605 (PK136; 1:200), CD3 BV650 (17A2; 1:300), CD8 APC-Cy7 (53 -6.7; 1:200), and CD4 PE-Cy5(GK1.5; 1:200) (Biolegend) antibodies. Intracellular staining was then performed using the Foxp3/transcription factor staining buffer set (eBioscience), where cells were fixed, permeabilized, and then stained with GZMB APC (GB11, Biolegend; 1:100), IFNγ V450 (XMG1.2, TONBO Biosciences; 1:100), and TNFα PE-Cy7 (MP6-XT22, eBiosciences; 1:100) antibodies. GZMB, IFNγ, and TNFα positivity was evaluated by gating on CD3⁻NK1.1⁺ NK cells and CD3⁺CD4⁺ and CD8⁺ T cells on an BD LSR II flow cytometer and analyzed using FlowJo (TreeStar) as described above.

### Pearson's Correlation Analysis

Gene expression data of primary PDAC patient tumors from two independent studies by Bailey et al. (GSE36924)(36) and Moffitt et al. (GSE71729)(37) were downloaded with the GEOquery2 package. Correlation analysis between NK (48) and T cell (49) gene signatures, STING (TMEM174) and TLR4 gene expression, and STING (50), TLR4, IRF3 (51), and IFNα/β signaling gene sets was performed using the ggpubr package. Results are presented as Pearson's correlation coefficient (R) values.

### Statistical Analysis

Statistical analyses were performed as described in the corresponding figure legends. Statistical significance was determined by two-sided Student's t-test, 1- or 2-way analysis of variance (ANOVA) with Tukey's post-tests, or log-rank test with Prism 10 software (GraphPad) and R. Values are reported as mean±standard error of at least 3 independent biological replicates, and sample numbers (n) are indicated in the figure legend. Significance was set at P<0.05.

### Example 1. NP Encapsulation Facilitates Safe and Effective Delivery of STING and TLR4 Agonists to the PDAC TME

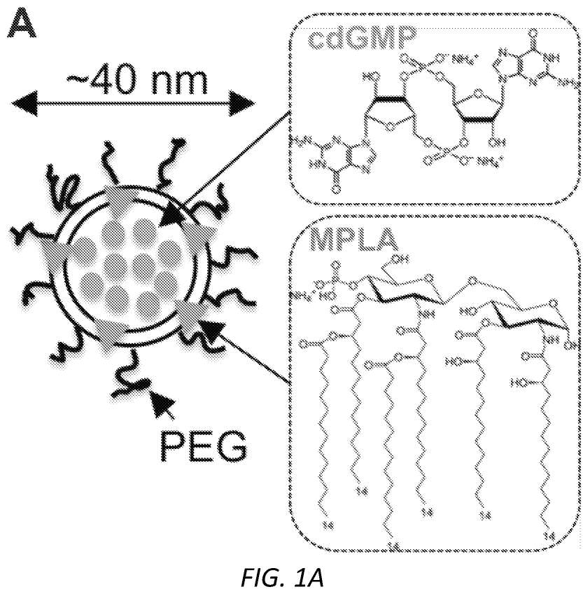

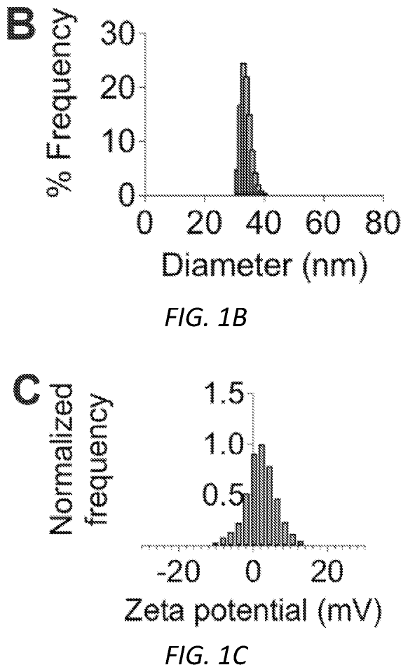

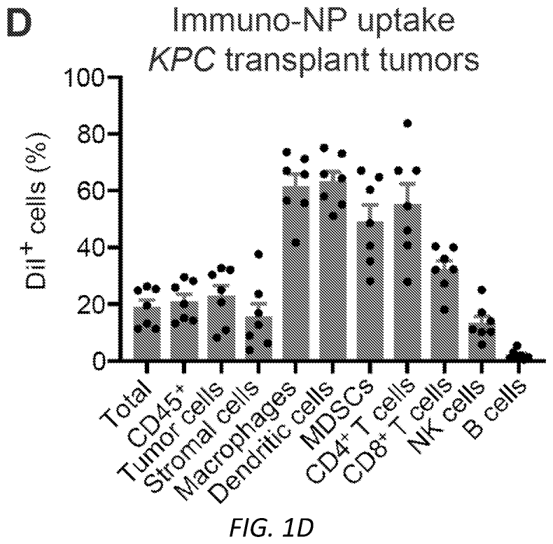

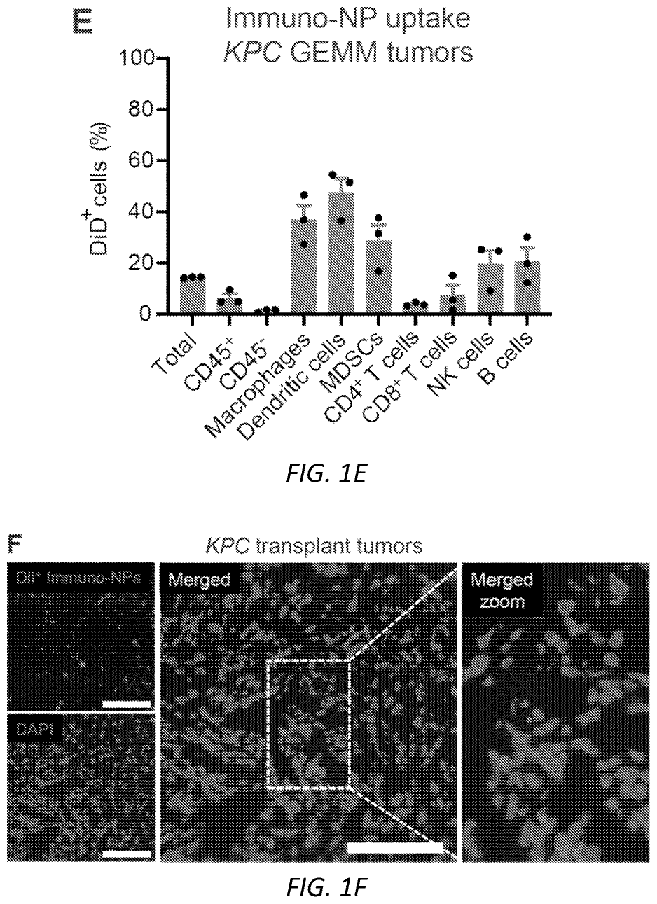

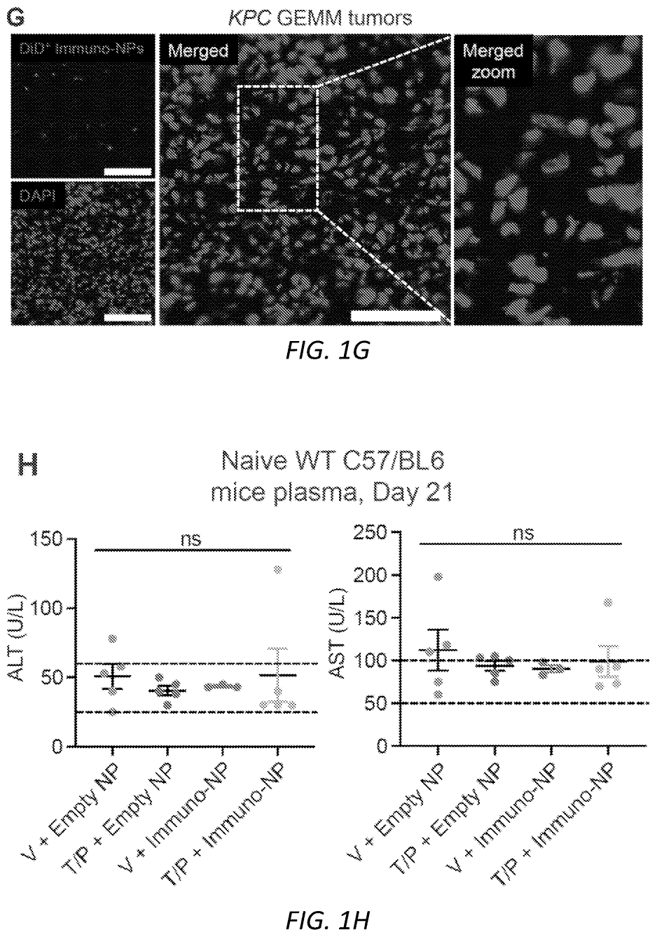

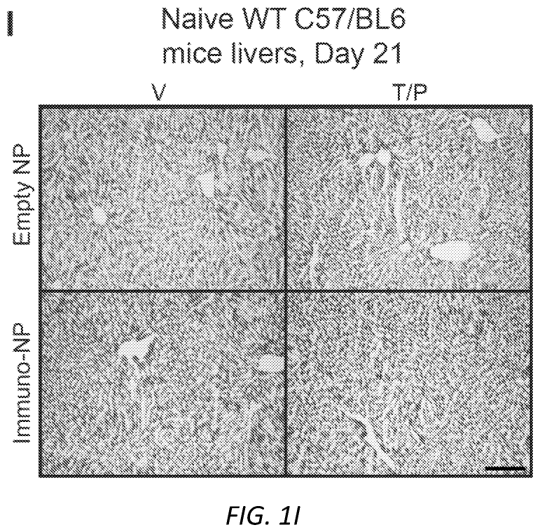

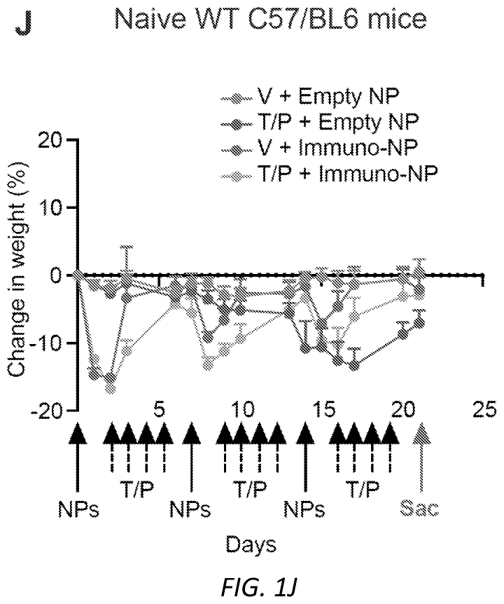

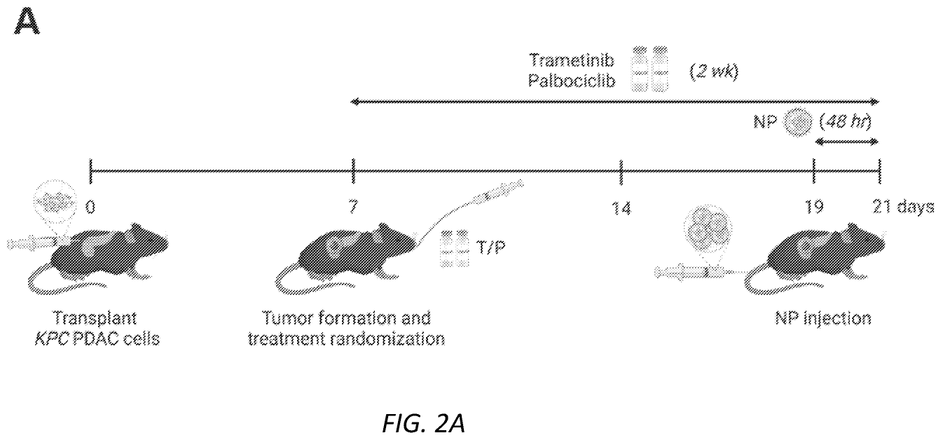

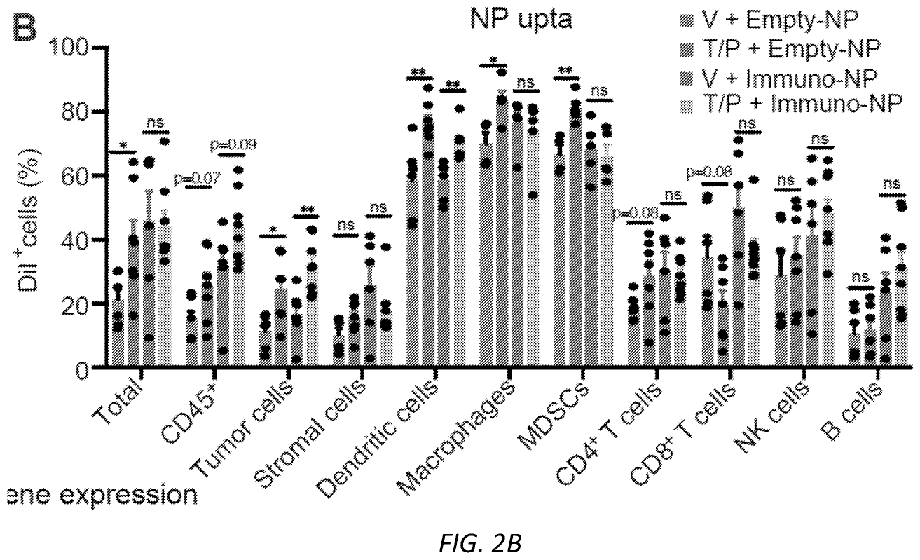

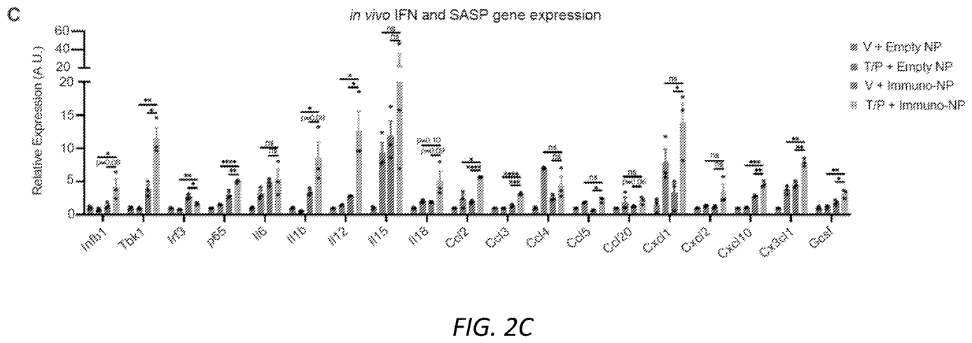

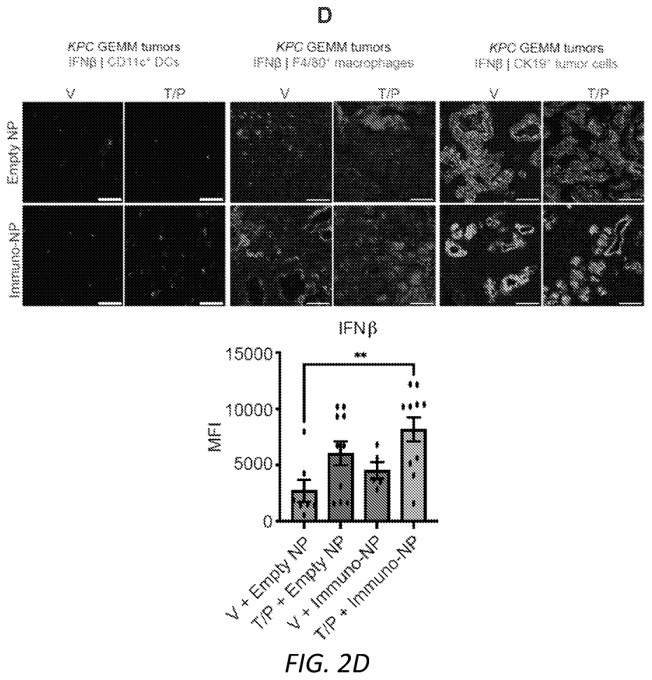

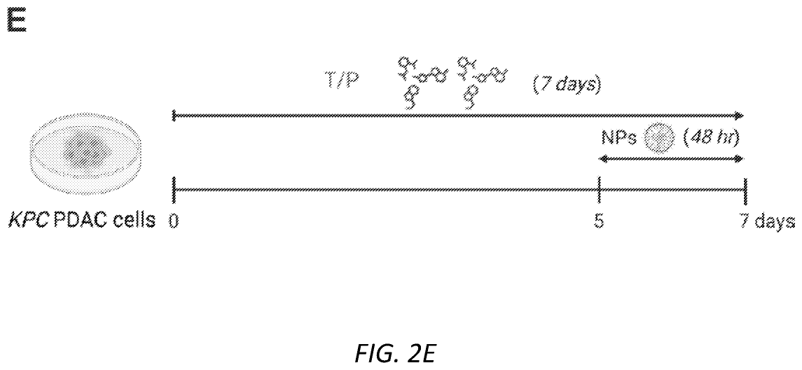

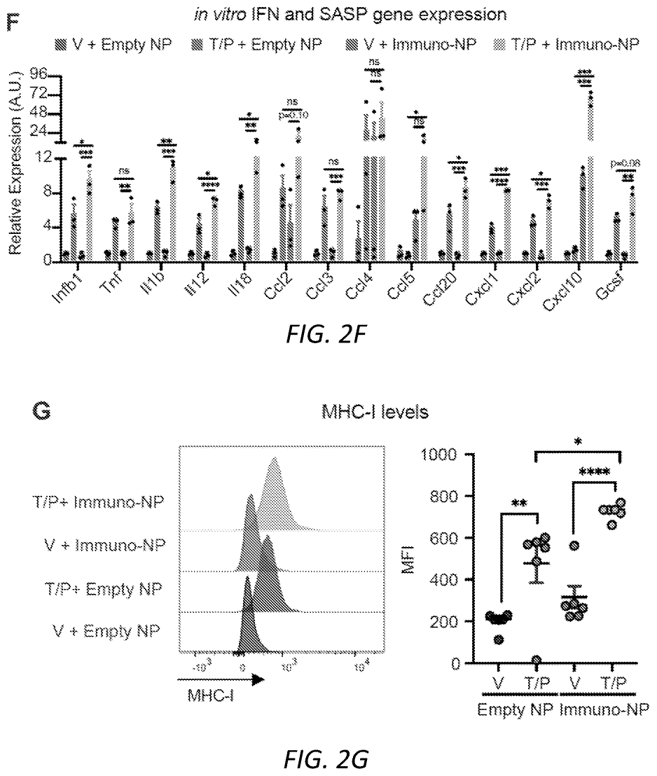

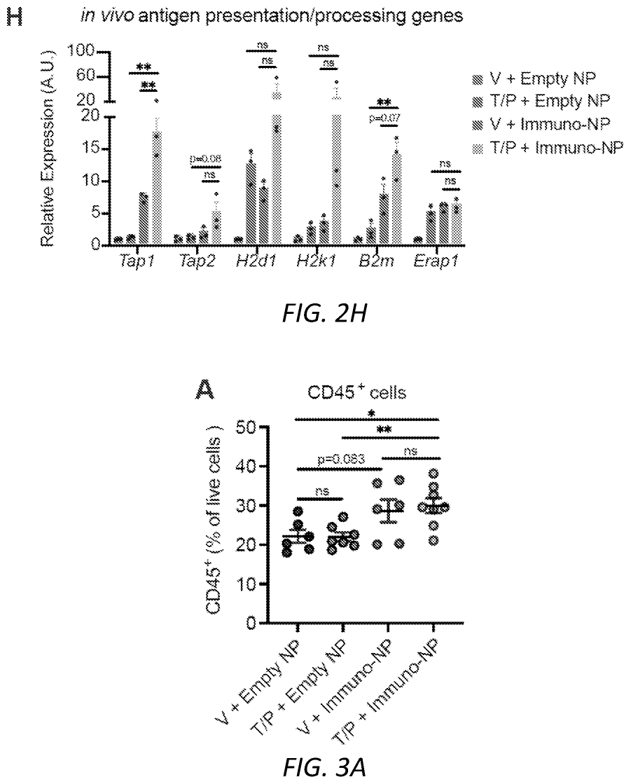

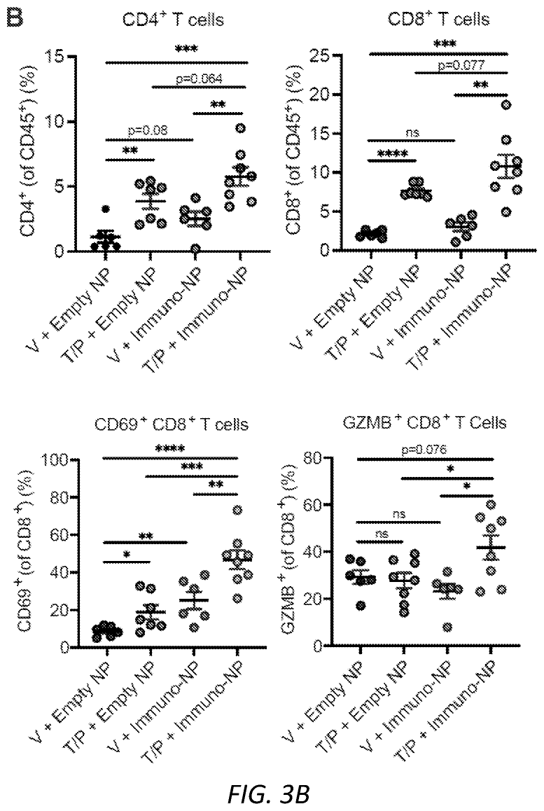

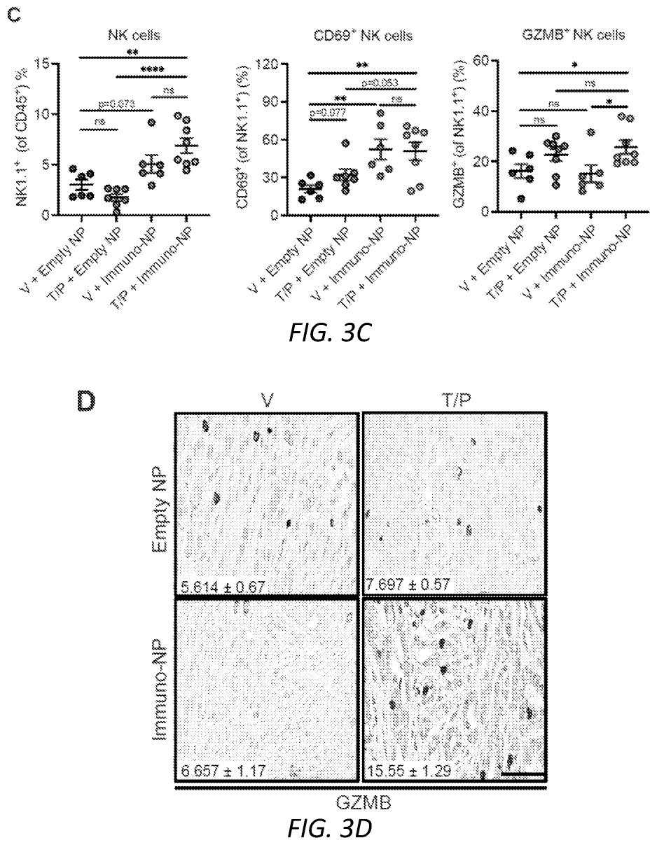

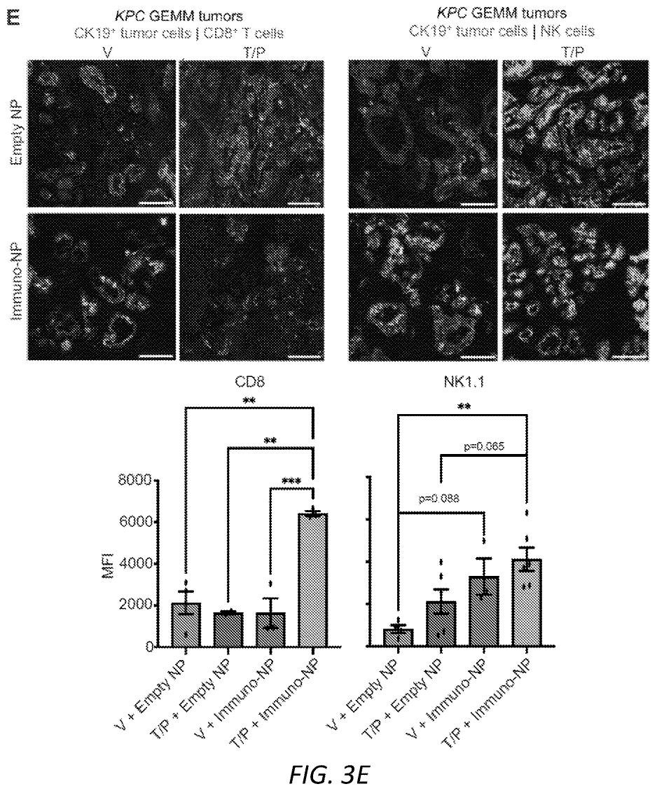

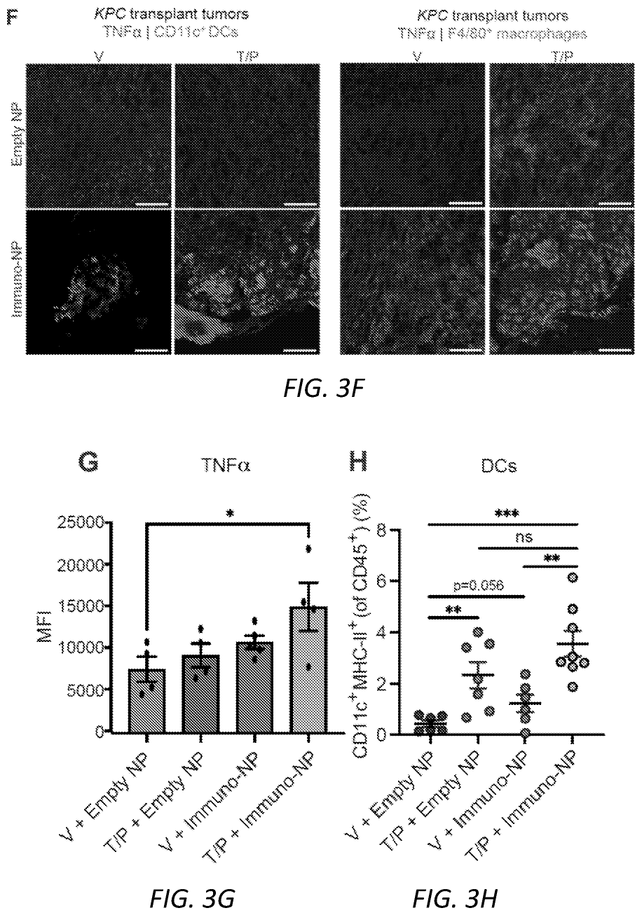

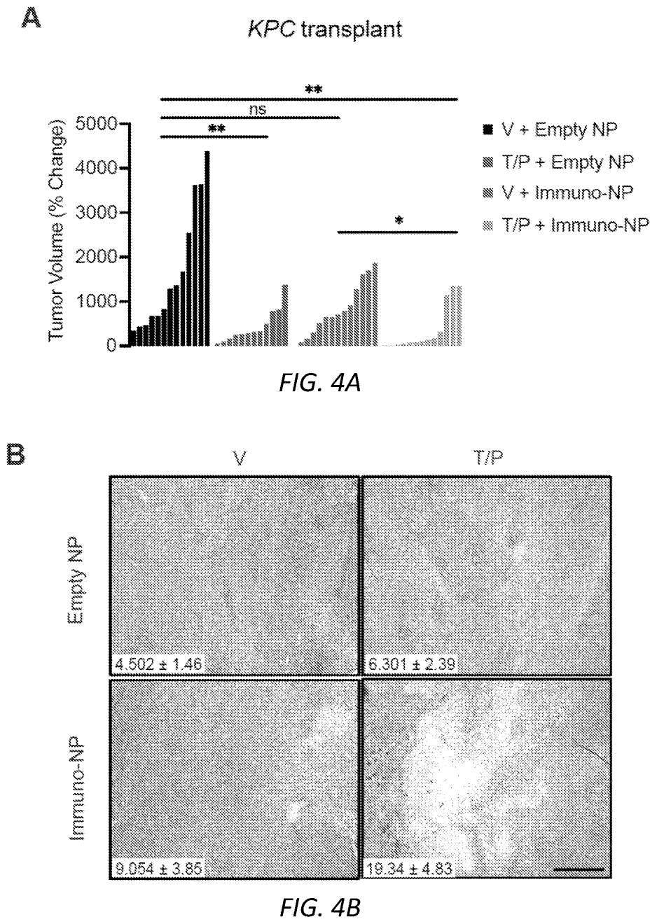

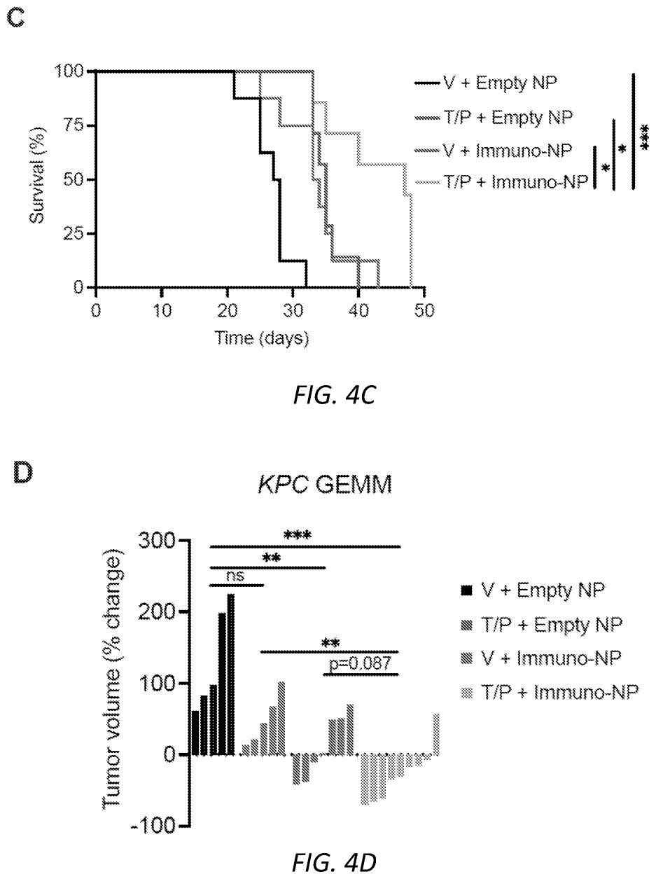

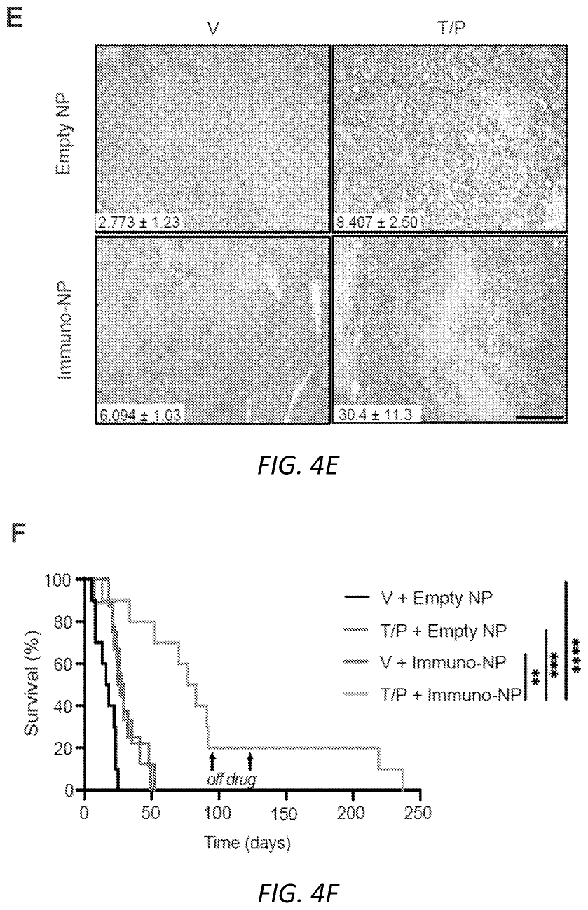

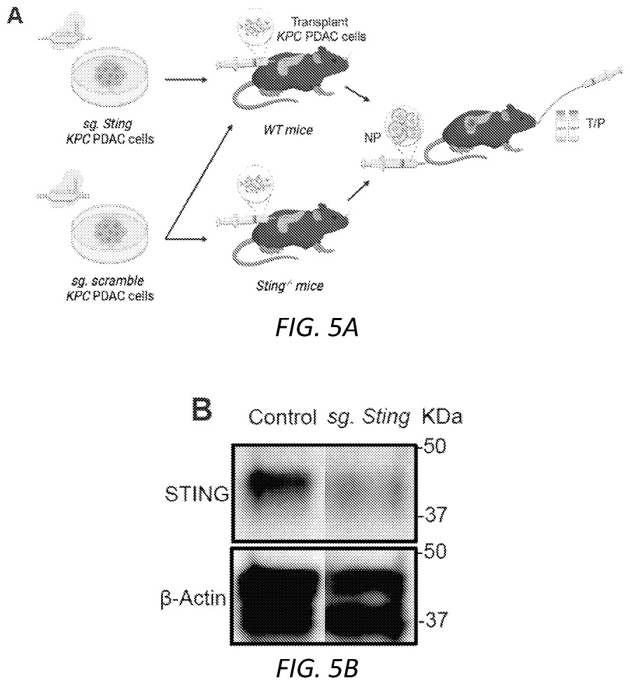

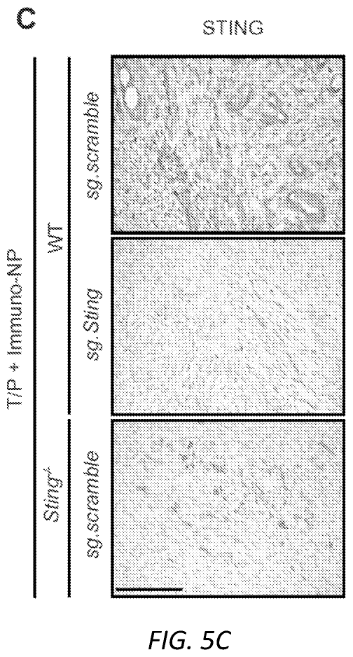

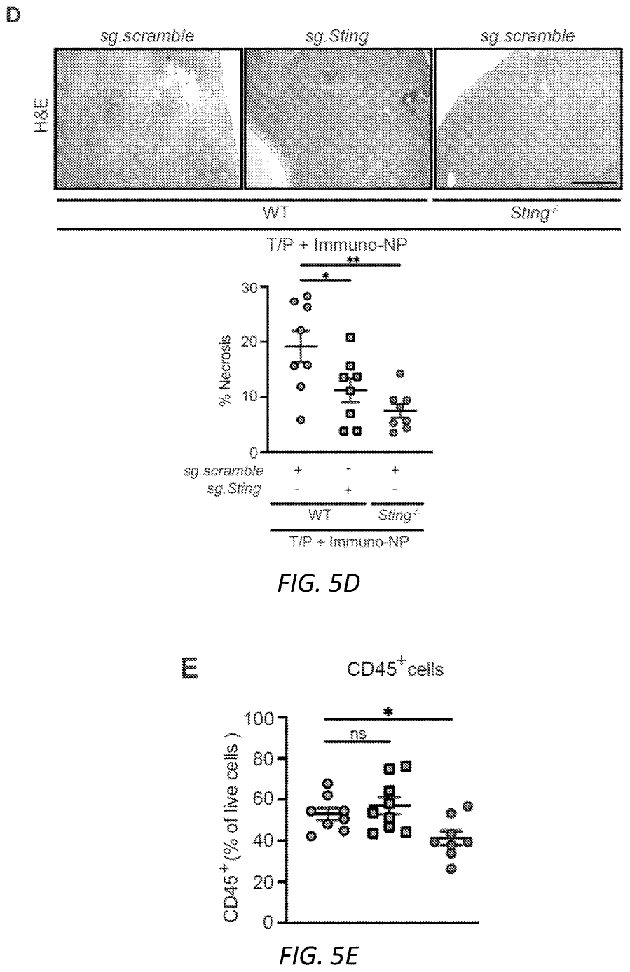

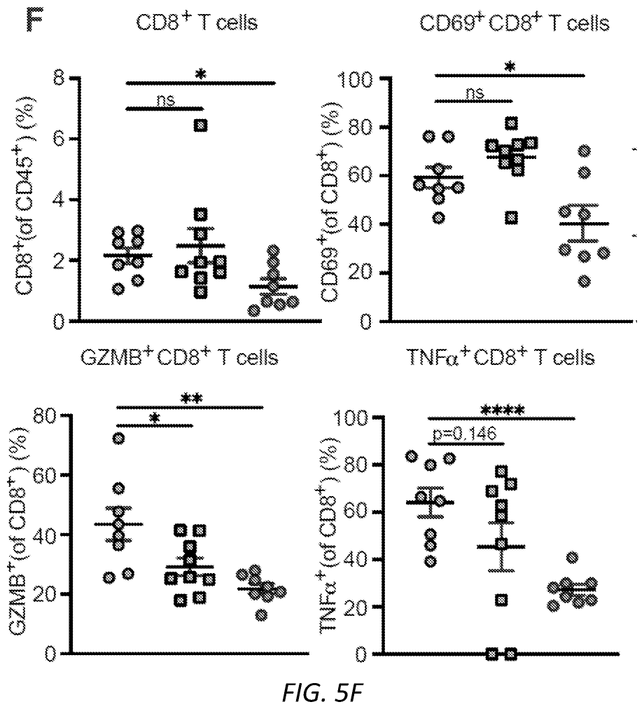

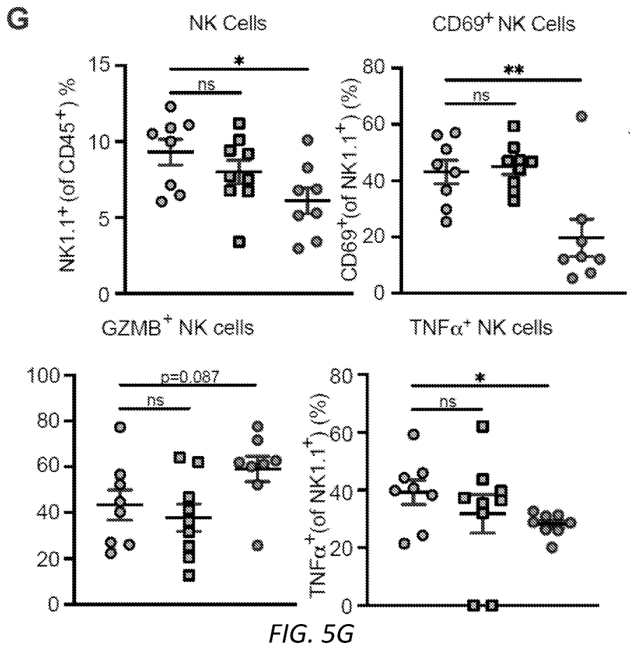

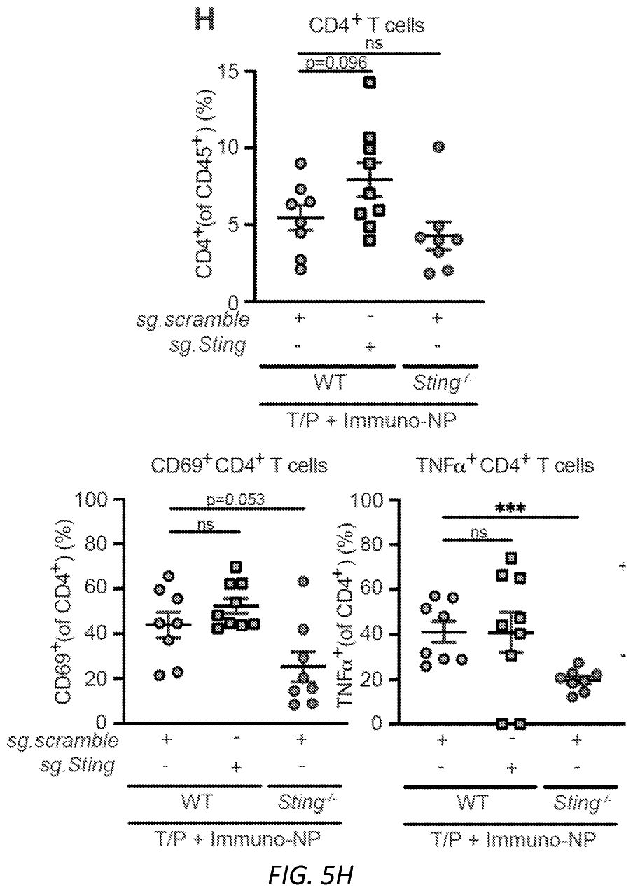

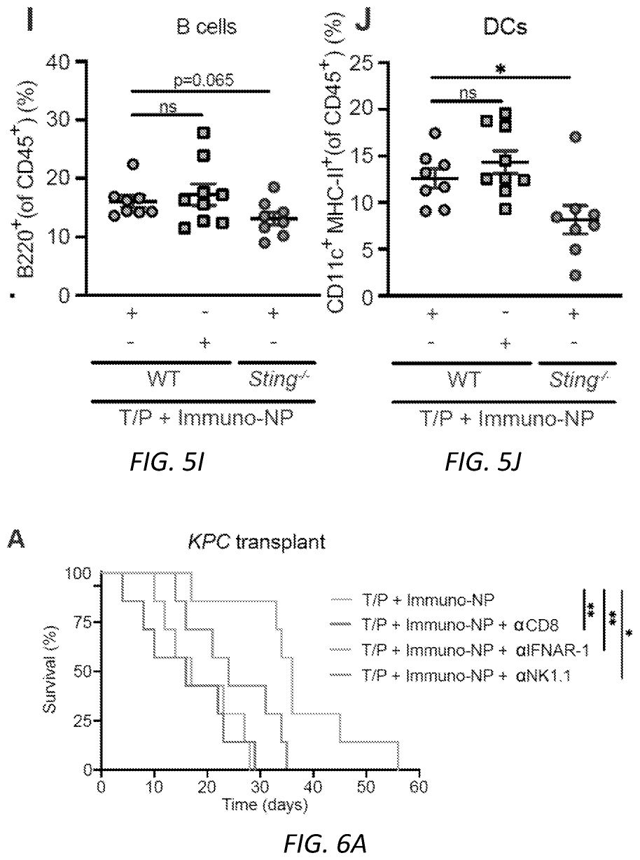

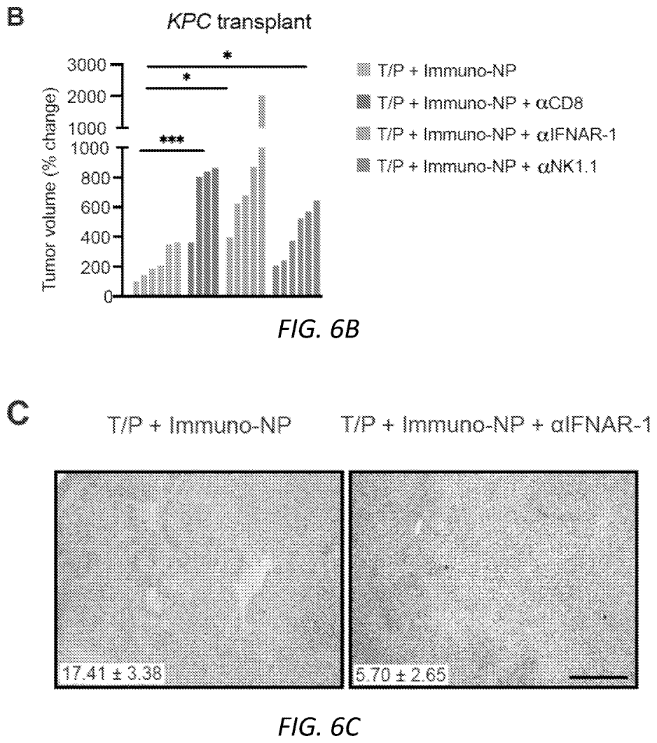

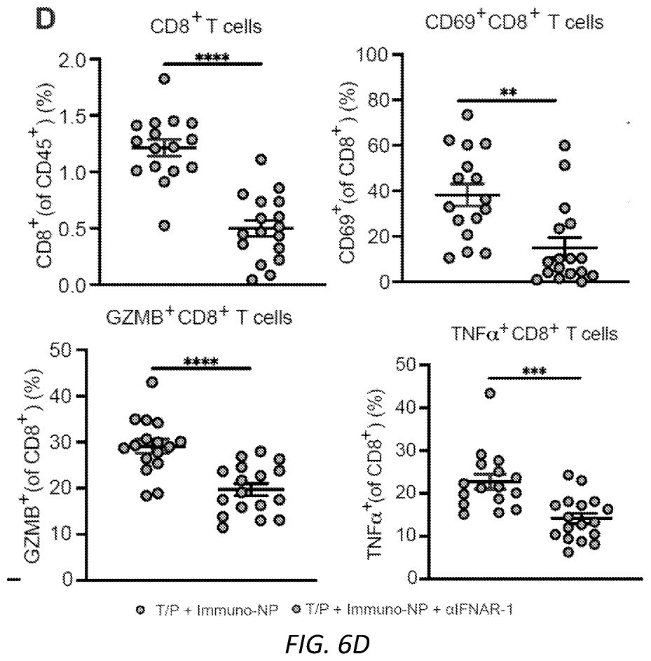

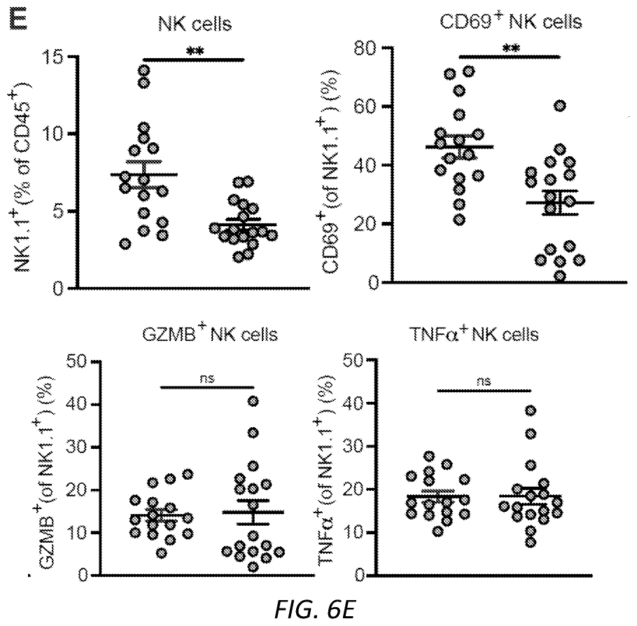

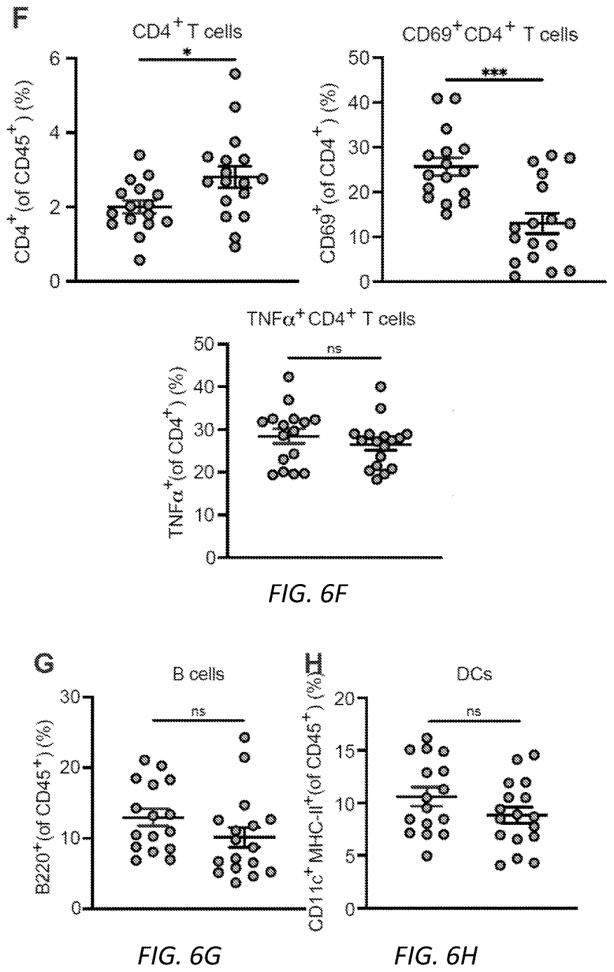

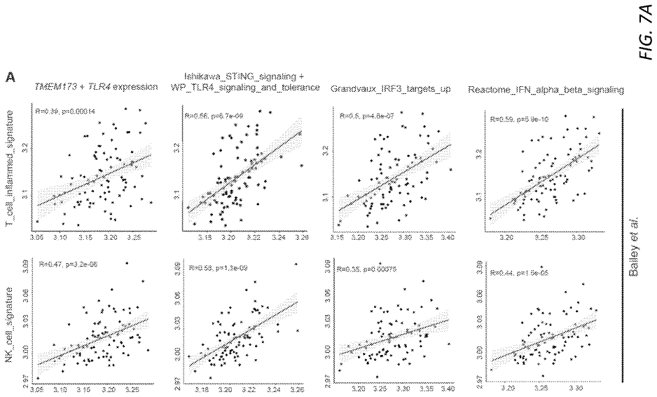

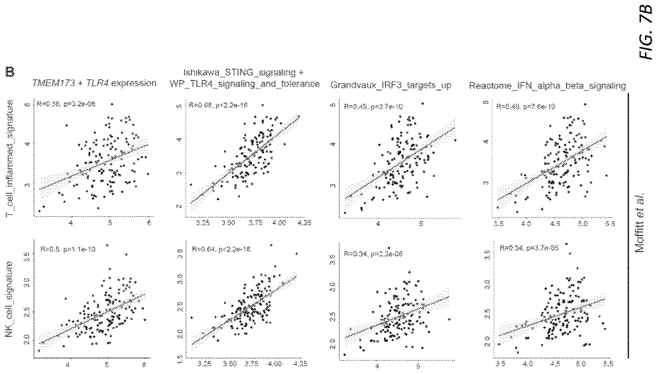

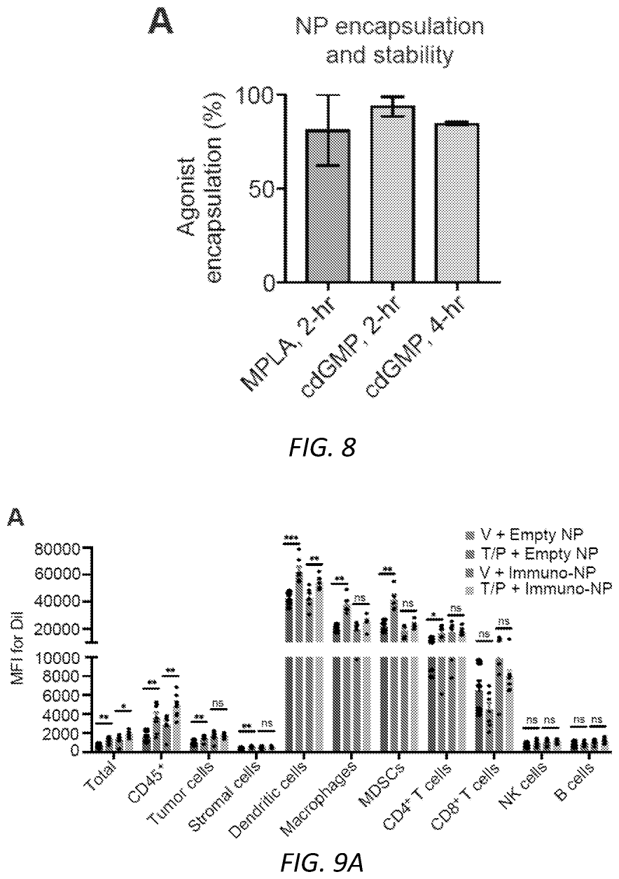

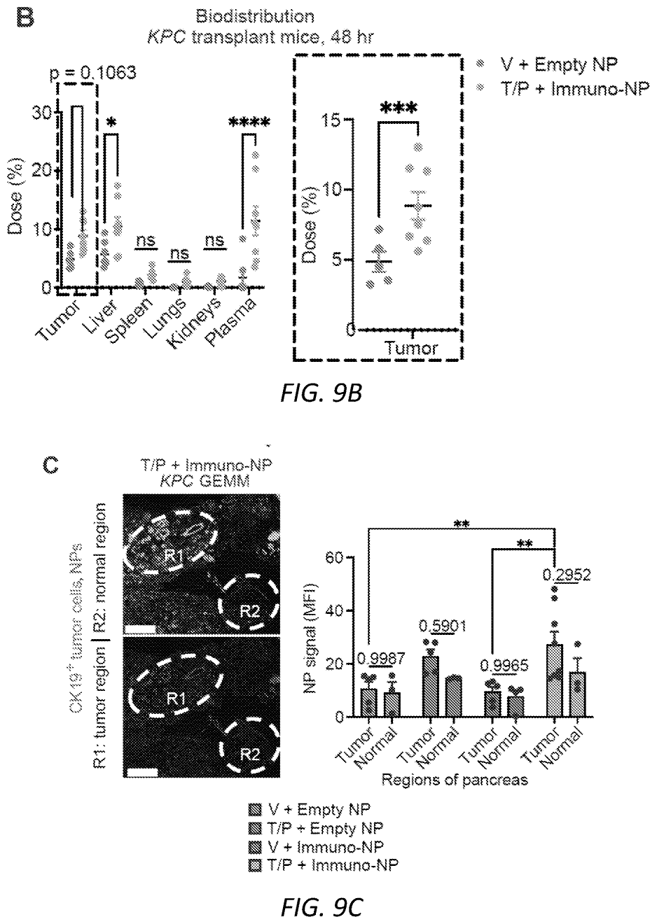

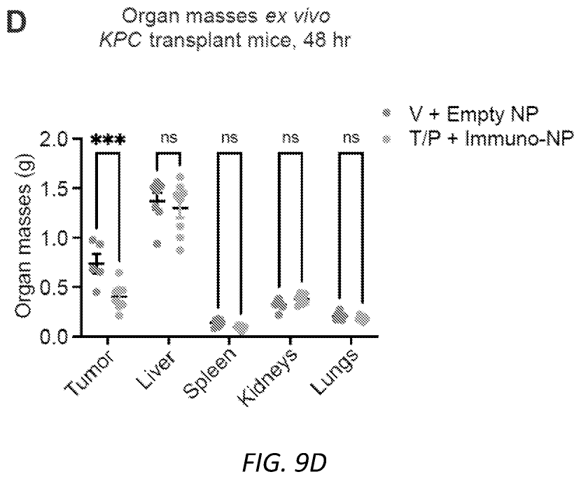

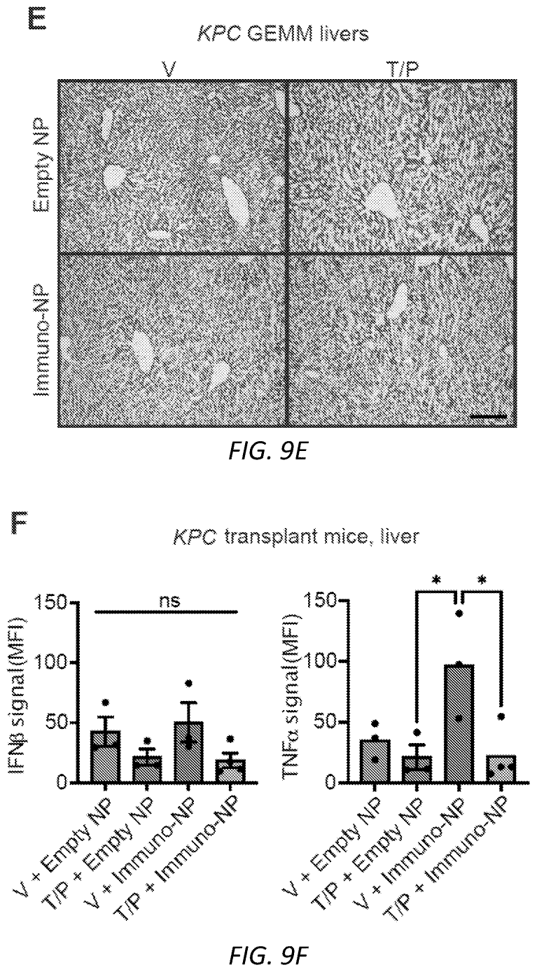

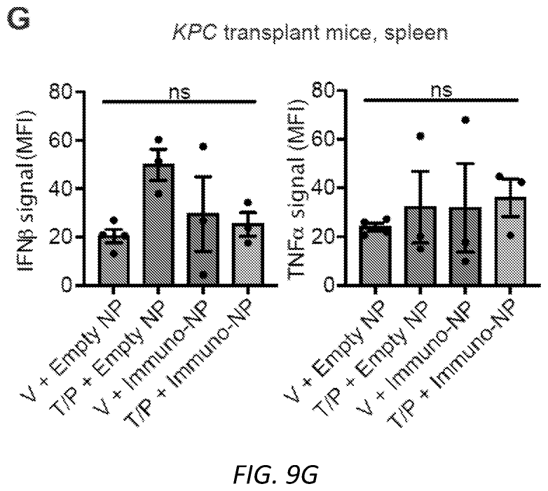

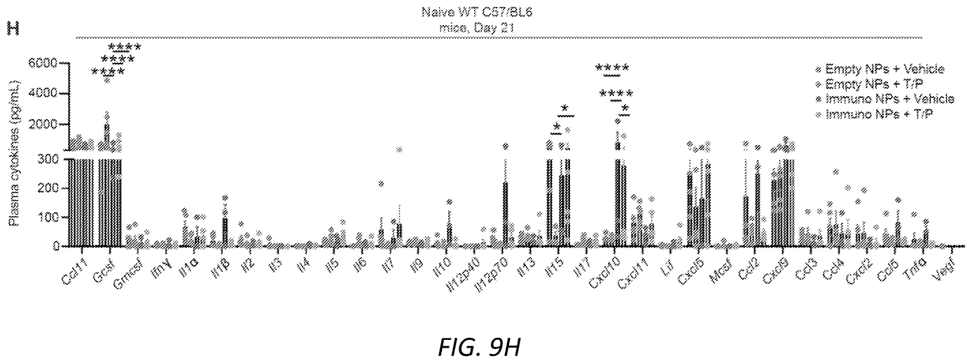

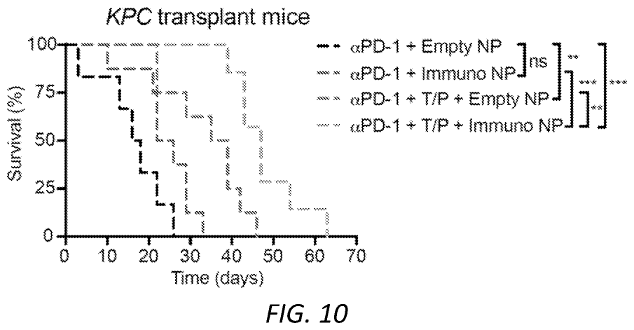

We first set out to assess whether STING and TLR4 agonists could be delivered to the PDAC TME through systemic intravenous (i.v.) administration following their co-encapsulation in lipid-based nanoparticles (immuno-NPs). Specifically, immuno-NPs were engineered with a ~40-nm diameter and “stealth” poly(ethylene) glycol (PEG) surface to enable delivery in the systemic blood circulation. These lipid-based materials supported the stable co-loading of the hydrophilic STING agonist, cdGMP, in the aqueous core and the hydrophobic TLR4 agonist, MPLA, in the lipid bilayer shell (FIG. 1, A to C and FIG. 8). A lipid fluorescent Di tracer was also incorporated into the bilayer to track and assess NP biodistribution in vivo. Fluorescent NPs were then administered systemically into the bloodstream by tail vein injection to either tumor-bearing (a) C57BL/6 mice orthotopically transplanted with KPC PDAC cell lines engineered with a luciferase-GFP reporter to track them in vivo or (b) P48-Cre; Kras^{LSL-G12D/wt}; Trp53^{fl/wt} (KPC) genetically engineered mouse models (GEMMs) that spontaneously develop PDAC.

NPs could be detected in the PDAC TME in both transplant and autochthonous PDAC models 48 hours post-injection. In KPC transplant mice, NPs were taken up by ~20% of live cells in the PDAC TME and preferentially, as predicted, by myeloid cells such as macrophages, DCs, and myeloid-derived suppressor cells (MDSCs) (FIGS. 1, D and F). Other immune cell populations, as well as tumor cells and CD45⁻ stromal cells, also stained positive for the fluorophore-labeled NPs, albeit to a lesser extent (FIG. 1D). NPs were also successfully delivered to myeloid cells and other immune populations in the densely fibrotic PDAC TME of KPC GEMMs that is notoriously difficult for drugs to penetrate (FIGS. 1, E and G).

We further measured NP biodistribution in other organs and in circulation of naïve Wild-type (WT) C57BL/6 mice and possible systemic toxicity following repeated dosing. Despite deposition of NPs to some degree in the liver where they are known to accumulate following i.v. administration (33), NPs did not accumulate in other highly vascularized organs such as the spleen, lung, or normal pancreas, and did not lead to observable liver toxicity as determined by AST/ALT levels or changes in histopathology (FIGS. 1, H and I). Moreover, while transient weight loss was observed 24-48 hours following NP administration, mice were able to quickly recover and regain their normal body weight within days (FIG. 1J). There were also no changes in organ masses following repeated NP dosing. Collectively, these results demonstrate that systemic administration of NPs can safely and effectively deliver immune stimulatory STING and TLR4 agonists to multiple cell types in the PDAC TME.

### Example 2. T/P Treatment Enhances Immuno-NP Delivery and in Combination Leads to Synergistic Induction of IFN Signaling and Antigen Presentation in PDAC

As we previously demonstrated that the MEK inhibitor trametinib and CDK4/6 inhibitor palbociclib (T/P) induce senescence and a pro-angiogenic SASP leading to vascular remodeling and increased drug uptake in PDAC lesions (13), we hypothesized that combining T/P with immuno-NP treatment would further enhance NP uptake and biodistribution in the TME. Indeed, pre-treatment with T/P for 12 days increased the uptake of immuno-and control unloaded (empty) NPs into PDAC lesions in KPC transplant mice 48 hours post-injection, particularly in tumor and myeloid (macrophage, DC) cell types (FIGS. 2, A and B, and FIG. 9A). NP biodistribution, as measured by Spectrum imaging of fluorescently-labeled NPs, was also enhanced following T/P administration locally in PDAC tumors as well as the liver and blood, but not in other organs such as the spleen, lungs, and kidneys (FIG. 9B). Remarkably, T/P pre-treatment increased NP deposition in CK19⁺ PDAC lesions but not adjacent normal areas in the same pancreas of tumor-bearing mice (FIG. 9C) or in the healthy pancreas of naïve non-tumor-bearing mice. Importantly, we did not observe any changes in markers of organ and blood toxicity or animal weights with combined T/P and immuno-NP treatment after short-term (48 hour) or repeated long-term NP dosing (3 weeks) in both tumor-bearing and naïve C57BL/6 mice (FIGS. 1, H to J, and 9, D and E).

Combined T/P and immuno-NP treatment significantly increased expression of not only downstream STING pathway components (Tbk1, Irf3) and the Type I interferon Ifnb1, but also pro-inflammatory SASP regulators (p65) and factors that we have previously shown to be repressed in the PDAC TME (15) compared to either treatment alone, including cytokines (Il12, Il18) and chemokines (Ccl2, Ccl3, Cxcl10, Cx3cl1) important for the activation and infiltration of cytotoxic NK and T lymphocytes (FIG. 2C). Importantly, we did not observe an increase in inflammatory markers in the blood stream, liver, or spleen following combined immuno-NP and T/P treatment, suggesting only a local inflammatory event in the PDAC TME induced by the therapy (FIG. 9, F to H). Co-immunofluorescence staining in both KPC transplant and GEMM PDAC lesions further revealed strong induction of IFNβ following T/P and immuno-NP treatment not just in APCs such as DCs and macrophages in the TME, but also in tumor cells where it is not normally expressed (FIG. 2D).

Given the unexpected induction of IFNβ as well as phosphorylation of its upstream regulator IRF3 (p-IRF3) within PDAC tumor cells, we investigated whether T/P and immuno-NP treatment also synergized in a tumor cell autonomous manner to further enhance pro-inflammatory SASP signaling. Combined treatment of murine KPC PDAC tumor cells in culture increased the phosphorylation of downstream STING signaling components IRF3 and p65, a subunit of NF-κB that we have shown to be a master transcriptional regulator of the pro-inflammatory SASP (34), and led to significant induction of interferon genes and SASP cytokines and chemokines (FIG. 2, E to F). This enhancement of interferon and inflammatory signaling upon dual T/P and immuno-NP treatment was also observed in another mouse pancreatic tumor cell line, Panc02, as well as human PANC-1 PDAC tumor cells. By contrast, though immuno-NPs alone induced expression of Ifnb1 and some cytokines (Il6, Il15) and chemokines (Ccl4) in RAW 264.7 macrophages, T/P treatment in combination did not enhance this effect. Combined T/P and immuno-NP treatment also significantly increased MHC-I surface protein levels on KPC PDAC cells in vitro, as well as antigen presentation/processing gene expression in the PDAC TME in vivo (FIG. 2, G to H). Taken together, these findings demonstrate that T/P and immuno-NP treatment synergize through both tumor cell autonomous and non-cell autonomous molecular mechanisms to enhance interferon and pro-inflammatory cytokine production and antigen presentation in the PDAC TME.

### Example 3. Immuno-NP and T/P Treatment Activates Cytotoxic NK and T Cell Immunity in PDAC Models

Given their synergistic effects on antigen presentation and interferon and cytokine signaling in the PDAC TME, we next investigated the impact of immuno-NP and T/P therapy on innate and adaptive immune responses in KPC PDAC transplant models and GEMMs by flow cytometry, immunofluorescence, and immunohistochemistry analysis. Similar to our previous findings, while a 2-week T/P treatment increased CD4⁺ and CD8⁺ T cell numbers and a single dose of immuno-NPs enhanced NK cell accumulation and proliferation (as marked by CD69) 48 hours later, neither of these single treatment arms alone was able to induce robust NK and T cell effector functions as assessed by induction of the degranulation marker Granzyme B (GZMB) or cytokines TNFα and IFNγ (FIG. 3, A to E). In contrast, combined immuno-NP and T/P treatment led not only to a further increase in CD4⁺ and CD8⁺ T cell and NK cell numbers and infiltration within tumor areas compared to each single treatment regimen, but also significantly enhanced the percentage of activated (CD69⁺) and cytotoxic (GZMB⁺) CD8⁺ T cells and IFNγ⁺ NK cells (FIG. 3, A to E).

Combined treatment also reduced immune suppressive cell populations, including B cells that were recently shown to mediate resistance to STING agonists by inhibiting NK cell functions in PDAC (25), as well as MDSCs that were enriched following T/P therapy alone. Moreover, whereas CD4⁺ T cell numbers and activation increased (FIG. 3B), FOXP3⁺ regulatory T cells (Tregs) that act to inhibit cytotoxic CD8⁺ T cell activity were severely reduced following combined treatment. In contrast, the amount of mature and activated TNFα⁺ macrophages and DCs, as well as MHC-II⁺ DCs that present antigen to T cells, expanded following dual NP and T/P therapy (FIG. 3, F to H). These immunological effects occurred locally in the PDAC TME, as there were no significant changes in total CD45⁺ immune cell numbers or innate DC, macrophage, and NK cell populations in the blood or other organs including the liver, spleen, lungs, and even normal pancreas of naïve WT mice treated with combined Immuno-NP and T/P regimens. Thus, combined immuno-NP and T/P treatment leads a reduction in suppressive Treg, MDSC, and B cell numbers, an increase in antigen-presenting DCs, and significantly enhanced NK and CD8⁺ T cell accumulation and activation locally in the PDAC TME.

### Example 4. Immuno-NP and T/P Therapy Leads to Tumor Regressions and Long-Term Survival in Preclinical PDAC Models

Combined immuno-NP and T/P treatment also mediated profound short-and long-term anti-tumor responses. Two-week treatment of PDAC-bearing KPC transplant mice with T/P four times per week and immuno-NPs weekly significantly reduced tumor growth compared to immuno-NP treatment alone (FIG. 4A). Moreover, T/P treatment for 2 weeks followed by a single dose of immuno-NPs produced large areas of tumor necrosis within just 48 hrs (FIG. 4B). This increased tumor control following dual immuno-NP and T/P therapy led to a significant improvement in the overall survival of PDAC-bearing KPC transplant mice compared to either single therapy alone following continuous treatment (FIG. 4C). The anti-tumor responses were even more striking in autochthonous KPC GEMM mice, where combined immuno-NP and T/P treatment led to tumor necrosis and shrinkage in 8/9 as compared to 3/7 or 0/5 animals treated with immuno-NP or T/P alone, respectively (FIGS. 4, D and E), as well as a significant long-term survival benefit (FIG. 4F). Remarkably, 20% of mice treated with immuno-NPs and T/P had complete tumor responses, though tumors eventually relapsed once treatment ceased (FIG. 4F). Together, these results demonstrate that combined immuno-NP and T/P therapy can produce long-term tumor control and potentially curative responses in preclinical PDAC models.

To investigate why tumors eventually relapsed off-treatment, we performed immunophenotyping on PDAC-bearing KPC transplant mice treated concurrently and continuously with NP and T/P regimens for two weeks. The enhanced accumulation of NK and T cells and their activation in the PDAC TME observed after acute 48-hour NP treatment (FIG. 3, A to E) was diminished after long-term 2-week treatment. In addition, the immune checkpoint PD-1 was highly induced on T cells and NK cells following both single as well as combined T/P and Immuno-NP treatment, indicating these cytotoxic lymphocytes may become exhausted from prolonged treatment and benefit from additional anti-PD-1 ICB. Indeed, a triple T/P, immuno-NP, and anti-PD-1 antibody (RMP1-14) regimen led to significantly increased survival in tumor-bearing KPC transplant mice compared to anti-PD-1 ICB alone or in combination with either NP or T/P regimens separately (FIG. 10). These results indicate that the addition of anti-PD-1 regimens can be used to prolong the anti-tumor effects of dual T/P and immuno-NP therapy.

### Example 5. Tumor and Host Sting Signaling Are Both Necessary for Anti-Tumor Immune Responses to Therapy

As immuno-NPs are taken up by both tumor and immune cells in the PDAC TME and activate downstream STING signaling that is further enhanced in combination with T/P (see FIGS. 2, B and D, FIG. 9A, and FIG. S3A), we next investigated the requirement of tumor and host STING signaling for the anti-tumor immune effects observed with dual NP and T/P therapy. To do so, we compared the outcomes of T/P and immuno-NP therapy in (a) control PDAC tumors transplanted into WT mice, Sting-deficient PDAC tumors transplanted into WT mice, or (c) control PDAC tumors transplanted into Sting^{-/-} mice (FIG. 5A). Both Sting knockout (KO) in KPC tumor cells using CRISPR/Cas9 editing and Sting KO in genetically engineered hosts led to a significant reduction in tumor necrosis following combined 2-week T/P and 48-hour immuno-NP treatment (FIG. 5, B to D). Though Sting loss in tumor cells led to some decrease in CD8⁺ T cell and NK cell activation markers, loss of host Sting signaling had a much more significant effect on mitigating T cell and NK cell infiltration and cytotoxicity, as well as MHC-II⁺ DC enrichment, following treatment (FIG. 5F-J). Thus, both tumor and host STING signaling contribute to cytotoxic NK and CD8⁺ T cell immunity and anti-tumor efficacy following dual T/P and NP treatment, with host STING signaling playing the more predominant role.

### Example 6. Type I IFN-Mediated NK and CD8⁺ T Cell Immunity Drives Treatment Efficacy

To assess whether the tumor responses and survival benefit observed upon dual immuno-NP and T/P treatment were indeed dependent on cytotoxic lymphocyte immunity, we used monoclonal antibodies targeting NK1.1 (PK136) and CD8 (2.43) to deplete NK and cytotoxic T cells, respectively. CD8⁺ T cell, and to a lesser but still significant extent NK cell depletion, both resulted in increased tumor growth and reduced the survival of PDAC-bearing KPC transplant mice treated with combination therapy (FIGS. 6, A and B). This suggests that NK and CD8⁺ T cells that we have shown to increase in number and become activated following therapy (FIG. 3, A to E) are necessary for its anti-tumor efficacy.

IFNβ that is synergistically induced downstream of STING in tumor and immune cells following immuno-NP and T/P treatment can activate cytotoxic NK and T cell immunity by binding to the Type I interferon receptor, IFNAR, expressed on these cells (35). To explore the role of Type I interferon signaling in anti-tumor NK and T cell immunity following therapy, we also treated mice with an IFNAR-1 neutralizing antibody. IFNAR blockade not only significantly reversed NK and CD8⁺ T cell infiltration and activation induced by immuno-NP and T/P treatment, but also mitigated tumor growth suppression and the survival benefit of treatment to a similar extent as CD8 depletion alone (FIG. 6, A to H). Similar experiments were also performed in Ifnar 1^{-/-} mice to determine if host IFNAR expression is critical for the anti-tumor immune effects of therapy. Indeed, KPC transplant PDAC tumors propagated in Ifnar 1^{-/-} mice also had diminished tumor necrosis and decreased CD 8⁺ T cell numbers and cytotoxicity markers compared to those propagated in WT hosts following treatment. Collectively, these findings demonstrate that Type I interferons induced upon combined immuno-NP and T/P treatment signal through IFNAR to initiate anti-tumor NK and CD8⁺ T cell responses that mediate immunological tumor control in PDAC models.

### Example 7. Sting and TLR4-Driven Type I IFN Signaling Associated with NK and T Cell Signatures in Human PDAC

To determine whether STING and TLR4 activity and downstream IFN signaling are associated with NK and T cell immunity in human PDAC, we performed analysis on patient samples from two separate published transcriptomic datasets (36, 37). STING (TMEM173) and TLR4 expression in patient tumors positively correlated with expression of NK and T cell signature genes in both datasets (FIG. 7). Moreover, high expression of downstream STING and TLR4 pathway components, as well as IRF3 target and Type I IFN signaling genes were also associated with increased NK and T cell transcripts. Taken together with our functional experiments in preclinical PDAC mouse models, these results suggest that potent activation of downstream Type I IFN signaling could be a promising therapeutic avenue to reactivate anti-tumor immunity in PDAC patients.

### REFERENCES

1. R. L. Siegel, A. N. Giaquinto, A. Jemal, Cancer statistics, 2024. CA Cancer J Clin 74, 12-49 (2024).
2. W. J. Ho, E. M. Jaffee, L. Zheng, The tumour microenvironment in pancreatic cancer-clinical challenges and opportunities. Nat Rev Clin Oncol 17, 527-540 (2020).

3 J. R. Brahmer, S. S. Tykodi, L. Q. Chow, W. J. Hwu, S. L. Topalian, P. Hwu, C. G. Drake, L. H. Camacho, J. Kauh, K. Odunsi, H. C. Pitot, O. Hamid, S. Bhatia, R. Martins, K. Eaton, S. Chen, T. M. Salay, S. Alaparthy, J. F. Grosso, A. J. Korman, S. M. Parker, S. Agrawal, S. M. Goldberg, D. M. Pardoll, A. Gupta, J. M. Wigginton, Safety and activity of anti-PD-L1 antibody in patients with advanced cancer. N Engl J Med 366, 2455-2465 (2012).
4. R. E. Royal, C. Levy, K. Turner, A. Mathur, M. Hughes, U.S. Kammula, R. M. Sherry, S. L. Topalian, J. C. Yang, I. Lowy, S. A. Rosenberg, Phase 2 trial of single agent Ipilimumab (anti-CTLA-4) for locally advanced or metastatic pancreatic adenocarcinoma. J Immunother 33, 828-833 (2010).
5. A. S. Bear, R. H. Vonderheide, M. H. O'Hara, Challenges and Opportunities for Pancreatic Cancer Immunotherapy. Cancer Cell 38, 788-802 (2020).
6. S. Hegde, V. E. Krisnawan, B. H. Herzog, C. Zuo, M. A. Breden, B. L. Knolhoff, G. D. Hogg, J. P. Tang, J. M. Baer, C. Mpoy, K. B. Lee, K. A. Alexander, B. E. Rogers, K. M. Murphy, W. G. Hawkins, R. C. Fields, C. J. DeSelm, J. K. Schwarz, D. G. DeNardo, Dendritic Cell Paucity Leads to Dysfunctional Immune Surveillance in Pancreatic Cancer. Cancer Cell 37, 289-307 e289 (2020).
7. N. Muthalagu, T. Monteverde, X. Raffo-Iraolagoitia, R. Wiesheu, D. Whyte, A. Hedley, S. Laing, B. Kruspig, R. Upstill-Goddard, R. Shaw, S. Neidler, C. Rink, S. A. Karim, K. Gyuraszova, C. Nixon, W. Clark, A. V. Biankin, L. M. Carlin, S. B. Coffelt, O. J. Sansom, J. P. Morton, D. J. Murphy, Repression of the Type I Interferon Pathway Underlies MYC-and KRAS-Dependent Evasion of NK and B Cells in Pancreatic Ductal Adenocarcinoma. Cancer Discov 10, 872-887 (2020).
8. J. Canon, K. Rex, A. Y. Saiki, C. Mohr, K. Cooke, D. Bagal, K. Gaida, T. Holt, C. G. Knutson, N. Koppada, B. A. Lanman, J. Werner, A. S. Rapaport, T. San Miguel, R. Ortiz, T. Osgood, J. R. Sun, X. Zhu, J. D. McCarter, L. P. Volak, B. E. Houk, M. G. Fakih, B. H. O'Neil, T. J. Price, G. S. Falchook, J. Desai, J. Kuo, R. Govindan, D. S. Hong, W. Ouyang, H. Henary, T. Arvedson, V. J. Cee, J. R. Lipford, The clinical KRAS(G12C) inhibitor AMG 510 drives anti-tumour immunity. Nature 575, 217-223 (2019).
9 A. Ribas, D. Lawrence, V. Atkinson, S. Agarwal, W. H. Miller, Jr., M. S. Carlino, R. Fisher, G. V. Long, F. S. Hodi, J. Tsoi, C. S. Grasso, B. Mookerjee, Q. Zhao, R. Ghori, B. H. Moreno, N. Ibrahim, O. Hamid, Combined BRAF and MEK inhibition with PD-1 blockade immunotherapy in BRAF-mutant melanoma. Nat Med 25, 936-940 (2019).
10. E. S. Knudsen, V. Kumarasamy, S. Chung, A. Ruiz, P. Vail, S. Tzetzo, J. Wu, R. Nambiar, J. Sivinski, S. S. Chauhan, M. Seshadri, S. I. Abrams, J. Wang, A. K. Witkiewicz, Targeting dual signalling pathways in concert with immune checkpoints for the treatment of pancreatic cancer. Gut 70, 127-138 (2021).
11. P. J. R. Ebert, J. Cheung, Y. Yang, E. McNamara, R. Hong, M. Moskalenko, S. E. Gould, H. Maecker, B. A. Irving, J. M. Kim, M. Belvin, I. Mellman, MAP Kinase Inhibition Promotes T Cell and Anti-tumor Activity in Combination with PD-L1 Checkpoint Blockade. Immunity 44, 609-621 (2016).
12. M. Ruscetti, J. Leibold, M. J. Bott, M. Fennell, A. Kulick, N. R. Salgado, C. C. Chen, Y. J. Ho, F. J. Sanchez-Rivera, J. Feucht, T. Baslan, S. Tian, H. A. Chen, P. B. Romesser, J. T. Poirier, C. M. Rudin, E. de Stanchina, E. Manchado, C. J. Sherr, S. W. Lowe, NK cell-mediated cytotoxicity contributes to tumor control by a cytostatic drug combination. Science 362, 1416-1422 (2018).
13. M. Ruscetti, J. P. t. Morris, R. Mezzadra, J. Russell, J. Leibold, P. B. Romesser, J. Simon, A. Kulick, Y. J. Ho, M. Fennell, J. Li, R. J. Norgard, J. E. Wilkinson, D. Alonso-Curbelo, R. Sridharan, D. A. Heller, E. de Stanchina, B. Z. Stanger, C. J. Sherr, S. W. Lowe, Senescence-Induced Vascular Remodeling Creates Therapeutic Vulnerabilities in Pancreas Cancer. Cell 181, 424-441 e421 (2020).
14. L. Chibaya, J. Snyder, M. Ruscetti, Senescence and the tumor-immune landscape: Implications for cancer immunotherapy. Semin Cancer Biol 86, 827-845 (2022).
15. L. Chibaya, K. C. Murphy, K. D. DeMarco, S. Gopalan, H. Liu, C. N. Parikh, Y. Lopez-Diaz, M. Faulkner, J. Li, J. P. t. Morris, Y. J. Ho, S. K. Chana, J. Simon, W. Luan, A. Kulick, E. de Stanchina, K. Simin, L. J. Zhu, T. G. Fazzio, S. W. Lowe, M. Ruscetti, EZH2 inhibition remodels the inflammatory senescence-associated secretory phenotype to potentiate pancreatic cancer immune surveillance. Nat Cancer 4, 872-892 (2023).
16. L. Corrales, S. M. McWhirter, T. W. Dubensky, Jr., T. F. Gajewski, The host STING pathway at the interface of cancer and immunity. The Journal of Clinical Investigation 126, 2404-2411 (2016).
17. A. Ablasser, Z. J. Chen, cGAS in action: Expanding roles in immunity and inflammation. Science 363, (2019).
18. M. Motwani, S. Pesiridis, K. A. Fitzgerald, DNA sensing by the cGAS-STING pathway in health and disease. Nat Rev Genet 20, 657-674 (2019).
19. S. Gluck, B. Guey, M. F. Gulen, K. Wolter, T. W. Kang, N. A. Schmacke, A. Bridgeman, J. Rehwinkel, L. Zender, A. Ablasser, Innate immune sensing of cytosolic chromatin fragments through cGAS promotes senescence. Nat Cell Biol 19, 1061-1070 (2017).
20. H. Yang, H. Wang, J. Ren, Q. Chen, Z. J. Chen, cGAS is essential for cellular senescence. Proceedings of the National Academy of Sciences 114, E4612-E4620 (2017).
21. E. P. Vonderhaar, N. S. Barnekow, D. McAllister, L. McOlash, M. A. Eid, M. J. Riese, V. L. Tarakanova, B. D. Johnson, M. B. Dwinell, STING Activated Tumor-Intrinsic Type I Interferon Signaling Promotes CXCR3 Dependent Antitumor Immunity in Pancreatic Cancer. Cellular and Molecular Gastroenterology and Hepatology 12, 41-58 (2021).
22. W. Jing, D. McAllister, E. P. Vonderhaar, K. Palen, M. J. Riese, J. Gershan, B. D. Johnson, M. B. Dwinell, STING agonist inflames the pancreatic cancer immune microenvironment and reduces tumor burden in mouse models. Journal for Immuno Therapy of Cancer 7, 115 (2019).
23. C. R. Ager, A. Boda, K. Rajapakshe, S. T. Lea, M. E. D. Francesco, P. Jayaprakash, R. B. Slay, B. Morrow, R. Prasad, M. A. Dean, C. R. Duffy, C. Coarfa, P. Jones, M. A. Curran, High potency STING agonists engage unique myeloid pathways to reverse pancreatic cancer immune privilege. Journal for Immuno Therapy of Cancer 9, e003246 (2021).
24. L. Motedayen Aval, J. E. Pease, R. Sharma, D. J. Pinato, Challenges and Opportunities in the Clinical Development of STING Agonists for Cancer Immunotherapy. J Clin Med 9, (2020).
25. S. Li, B. Mirlekar, B. M. Johnson, W. J. Brickey, J. A. Wrobel, N. Yang, D. Song, S. Entwistle, X. Tan, M. Deng, Y. Cui, W. Li, B. G. Vincent, M. Gale, Y. Pylayeva-Gupta, J. P. Y. Ting, STING-induced regulatory B cells compromise NK function in cancer immunity. Nature 610, 373-380 (2022).
26. R. D. Luteijn, S. A. Zaver, B. G. Gowen, S. K. Wyman, N. E. Garelis, L. Onia, S. M. McWhirter, G. E. Katibah, J. E. Corn, J. J. Woodward, D. H. Raulet, SLC19A1 transports immunoreactive cyclic dinucleotides. Nature 573, 434-438 (2019).
27. P. U. Atukorale, S. P. Raghunathan, V. Raguveer, T. J. Moon, C. Zheng, P. A. Bielecki, M. L. Wiese, A. L. Goldberg, G. Covarrubias, C. J. Hoimes, E. Karathanasis, Nanoparticle Encapsulation of Synergistic Immune Agonists Enables Systemic Codelivery to Tumor Sites and IFNbeta-Driven Antitumor Immunity. Cancer Res 79, 5394-5406 (2019).
28. V. Mata-Haro, C. Cekic, M. Martin, P. M. Chilton, C. R. Casella, T. C. Mitchell, The vaccine adjuvant monophosphoryl lipid A as a TRIF-biased agonist of TLR4. Science 316, 1628-1632 (2007).
29. L. Sun, T. Kees, A. S. Almeida, B. Liu, X.-Y. He, D. Ng, X. Han, D. L. Spector, I. A. McNeish, P. Gimotty, S. Adams, M. Egeblad, Activating a collaborative innate-adaptive immune response to control metastasis. Cancer Cell 39, 1361-1374.e1369 (2021).
30. P. U. Atukorale, T. J. Moon, A. R. Bokatch, C. F. Lusi, J. T. Routhier, V. J. Deng, E. Karathanasis, Dual agonist immunostimulatory nanoparticles combine with PD1 blockade for curative neoadjuvant immunotherapy of aggressive cancers. Nanoscale 14, 1144-1159 (2022).
31. M. E. Lorkowski, P. U. Atukorale, P. A. Bielecki, K. H. Tong, G. Covarrubias, Y. Zhang, G. Loutrianakis, T. J. Moon, A. R. Santulli, W. M. Becicka, E. Karathanasis, Immunostimulatory nanoparticle incorporating two immune agonists for the treatment of pancreatic tumors. Journal of Controlled Release 330, 1095-1105 (2021).
32. G. Covarrubias, T. J. Moon, G. Loutrianakis, H. M. Sims, M. P. Umapathy, M. E. Lorkowski, P. A. Bielecki, M. L. Wiese, P. U. Atukorale, E. Karathanasis, Comparison of the uptake of untargeted and targeted immunostimulatory nanoparticles by immune cells in the microenvironment of metastatic breast cancer. J Mater Chem B 10, 224-235 (2022).
33. W. Ngo, S. Ahmed, C. Blackadar, B. Bussin, Q. Ji, S. M. Mladjenovic, Z. Sepahi, W. C. W. Chan, Why nanoparticles prefer liver macrophage cell uptake in vivo. Adv Drug Deliv Rev 185, 114238 (2022).
34. Y. Chien, C. Scuoppo, X. Wang, X. Fang, B. Balgley, J. E. Bolden, P. Premsrirut, W. Luo, A. Chicas, C. S. Lee, S. C. Kogan, S. W. Lowe, Control of the senescence-associated secretory phenotype by NF-kappaB promotes senescence and enhances chemosensitivity. Genes Dev 25, 2125-2136 (2011).
35. L. Zitvogel, L. Galluzzi, O. Kepp, M. J. Smyth, G. Kroemer, Type I interferons in anticancer immunity. Nat Rev Immunol 15, 405-414 (2015).
36. P. Bailey, D. K. Chang, K. Nones, A. L. Johns, A. M. Patch, M. C. Gingras, D. K. Miller, A. N. Christ, T. J. Bruxner, M. C. Quinn, C. Nourse, L. C. Murtaugh, I. Harliwong, S. Idrisoglu, S. Manning, E. Nourbakhsh, S. Wani, L. Fink, O. Holmes, V. Chin, M. J. Anderson, S. Kazakoff, C. Leonard, F. Newell, N. Waddell, S. Wood, Q. Xu, P. J. Wilson, N. Cloonan, K. S. Kassahn, D. Taylor, K. Quek, A. Robertson, L. Pantano, L. Mincarelli, L. N. Sanchez, L. Evers, J. Wu, M. Pinese, M. J. Cowley, M. D. Jones, E. K. Colvin, A. M. Nagrial, E. S. Humphrey, L. A. Chantrill, A. Mawson, J. Humphris, A. Chou, M. Pajic, C. J. Scarlett, A. V. Pinho, M. Giry-Laterriere, I. Rooman, J. S. Samra, J. G. Kench, J. A. Lovell, N. D. Merrett, C. W. Toon, K. Epari, N. Q. Nguyen, A. Barbour, N. Zeps, K. Moran-Jones, N. B. Jamieson, J. S. Graham, F. Duthie, K. Oien, J. Hair, R. Grutzmann, A. Maitra, C. A. Iacobuzio-Donahue, C. L. Wolfgang, R. A. Morgan, R. T. Lawlor, V. Corbo, C. Bassi, B. Rusev, P. Capelli, R. Salvia, G. Tortora, D. Mukhopadhyay, G. M. Petersen, I. Australian Pancreatic Cancer Genome, D. M. Munzy, W. E. Fisher, S. A. Karim, J. R. Eshleman, R. H. Hruban, C. Pilarsky, J. P. Morton, O. J. Sansom, A. Scarpa, E. A. Musgrove, U. M. Bailey, O. Hofmann, R. L. Sutherland, D. A. Wheeler, A. J. Gill, R. A. Gibbs, J. V. Pearson, N. Waddell, A. V. Biankin, S. M. Grimmond, Genomic analyses identify molecular subtypes of pancreatic cancer. Nature 531, 47-52 (2016).
37. R. A. Moffitt, R. Marayati, E. L. Flate, K. E. Volmar, S. G. Loeza, K. A. Hoadley, N. U. Rashid, L. A. Williams, S. C. Eaton, A. H. Chung, J. K. Smyla, J. M. Anderson, H. J. Kim, D. J. Bentrem, M. S. Talamonti, C. A. Iacobuzio-Donahue, M. A. Hollingsworth, J. J. Yeh, Virtual microdissection identifies distinct tumor-and stroma-specific subtypes of pancreatic ductal adenocarcinoma. Nat Genet 47, 1168-1178 (2015).
38. L. A. Rojas, Z. Sethna, K. C. Soares, C. Olcese, N. Pang, E. Patterson, J. Lihm, N. Ceglia, P. Guasp, A. Chu, R. Yu, A. K. Chandra, T. Waters, J. Ruan, M. Amisaki, A. Zebboudj, Z. Odgerel, G. Payne, E. Derhovanessian, F. Muller, I. Rhee, M. Yadav, A. Dobrin, M. Sadelain, M. Luksza, N. Cohen, L. Tang, O. Basturk, M. Gonen, S. Katz, R. K. Do, A. S. Epstein, P. Momtaz, W. Park, R. Sugarman, A. M. Varghese, E. Won, A. Desai, A. C. Wei, M. I. D'Angelica, T. P. Kingham, I. Mellman, T. Merghoub, J. D. Wolchok, U. Sahin, O. Tureci, B. D. Greenbaum, W. R. Jarnagin, J. Drebin, E. M. O'Reilly, V. P. Balachandran, Personalized RNA neoantigen vaccines stimulate T cells in pancreatic cancer. Nature 618, 144-150 (2023).
39. L. J. Padron, D. M. Maurer, M. H. O'Hara, E. M. O'Reilly, R. A. Wolff, Z. A. Wainberg, A. H. Ko, G. Fisher, O. Rahma, J. P. Lyman, C. R. Cabanski, J. X. Yu, S. M. Pfeiffer, M. Spasic, J. Xu, P. F. Gherardini, J. Karakunnel, R. Mick, C. Alanio, K. T. Byrne, T. J. Hollmann, J. S. Moore, D. D. Jones, M. Tognetti, R. O. Chen, X. Yang, L. Salvador, E. J. Wherry, U. Dugan, J. O'Donnell-Tormey, L. H. Butterfield, V. M. Hubbard-Lucey, R. Ibrahim, J. Fairchild, S. Bucktrout, T. M. La Vallee, R. H. Vonderheide, Sotigalimab and/or nivolumab with chemotherapy in first-line metastatic pancreatic cancer: clinical and immunologic analyses from the randomized phase 2 PRINCE trial. Nat Med 28, 1167-1177 (2022).
40. X. Liu, G. D. Hogg, C. Zuo, N. C. Borcherding, J. M. Baer, V. E. Lander, L. I. Kang, B. L. Knolhoff, F. Ahmad, R. E. Osterhout, A. V. Galkin, J. M. Bruey, L. L. Carter, C. Mpoy, K. R. Vij, R. C. Fields, J. K. Schwarz, H. Park, V. Gupta, D. G. DeNardo, Context-dependent activation of STING-interferon signaling by CD11b agonists enhances anti-tumor immunity. Cancer Cell 41, 1073-1090 e1012 (2023).
41. V. E. Lander, J. I. Belle, N. L. Kingston, J. M. Herndon, G. D. Hogg, X. Liu, L. I. Kang, B. L. Knolhoff, S. J. Bogner, J. M. Baer, C. Zuo, N. C.

Borcherding, D. P. Lander, C. Mpoy, J. Scott, M. Zahner, B. E. Rogers, J. K. Schwarz, H. Kim, D. G. DeNardo, Stromal Reprogramming by FAK Inhibition Overcomes Radiation Resistance to Allow for Immune Priming and Response to Checkpoint Blockade. Cancer Discov 12, 2774-2799 (2022).
42. B. Bockorny, V. Semenisty, T. Macarulla, E. Borazanci, B. M. Wolpin, S. M. Stemmer, T. Golan, R. Geva, M. J. Borad, K. S. Pedersen, J. O. Park, R. A.

Ramirez, D. G. Abad, J. Feliu, A. Munoz, M. Ponz-Sarvise, A. Peled, T. M. Lustig, O. Bohana-Kashtan, S. M. Shaw, E. Sorani, M. Chaney, S. Kadosh, A. Vainstein Haras, D. D. Von Hoff, M. Hidalgo, BL-8040, a CXCR4 antagonist, in combination with pembrolizumab and chemotherapy for pancreatic cancer: the COMBAT trial. Nat Med 26, 878-885 (2020).
43. L. Zhang, X. Wei, Z. Wang, P. Liu, Y. Hou, Y. Xu, H. Su, M. D. Koci, H. Yin, C. Zhang, NF-kappaB activation enhances STING signaling by altering microtubule-mediated STING trafficking. Cell Rep 42, 112185 (2023).
44. J. P. Coppe, P. Y. Desprez, A. Krtolica, J. Campisi, The senescence-associated secretory phenotype: the dark side of tumor suppression. Annu Rev Pathol 5, 99-118 (2010).
45. J. H. Strickler, H. Satake, T. J. George, R. Yaeger, A. Hollebecque, I. Garrido-Laguna, M. Schuler, T. F. Burns, A. L. Coveler, G. S. Falchook, M. Vincent, Y. Sunakawa, L. Dahan, D. Bajor, S. Y. Rha, C. Lemech, D. Juric, M. Rehn, G. Ngarmchamnanrith, P. Jafarinasabian, Q. Tran, D. S. Hong, Sotorasib in KRAS p. G12C-Mutated Advanced Pancreatic Cancer. N Engl J Med 388, 33-43 (2023).
46. S. B. Kemp, N. Cheng, N. Markosyan, R. Sor, I. K. Kim, J. Hallin, J. Shoush, L. Quinones, N. V. Brown, J. B. Bassett, N. Joshi, S. Yuan, M. Smith, W. P. Vostrejs, K. Z. Perez-Vale, B. Kahn, F. Mo, T. R. Donahue, C. G. Radu, C. Clendenin, J. G. Christensen, R. H. Vonderheide, B. Z. Stanger, Efficacy of a Small-Molecule Inhibitor of KrasG12D in Immunocompetent Models of Pancreatic Cancer. Cancer Discov 13, 298-311 (2023).
47. J. R. Infante, B. G. Somer, J. O. Park, C. P. Li, M. E. Scheulen, S. M. Kasubhai, D. Y. Oh, Y. Liu, S. Redhu, K. Steplewski, N. Le, A randomised, double-blind, placebo-controlled trial of trametinib, an oral MEK inhibitor, in combination with gemcitabine for patients with untreated metastatic adenocarcinoma of the pancreas. Eur J Cancer 50, 2072-2081 (2014).
48. J. Cursons, F. Souza-Fonseca-Guimaraes, M. Foroutan, A. Anderson, F. Hollande, S. Hediyeh-Zadeh, A. Behren, N. D. Huntington, M. J. Davis, A Gene Signature Predicting Natural Killer Cell Infiltration and Improved Survival in Melanoma Patients. Cancer Immunol Res 7, 1162-1174 (2019).
49. M. Ayers, J. Lunceford, M. Nebozhyn, E. Murphy, A. Loboda, D. R. Kaufman, A. Albright, J. D. Cheng, S. P. Kang, V. Shankaran, S. A. Piha-Paul, J. Yearley, T. Y. Seiwert, A. Ribas, T. K. McClanahan, IFN-gamma-related mRNA profile predicts clinical response to PD-1 blockade. J Clin Invest 127, 2930-2940 (2017).
50. H. Ishikawa, G. N. Barber, STING is an endoplasmic reticulum adaptor that facilitates innate immune signalling. Nature 455, 674-678 (2008).
51. N. Grandvaux, M. J. Servant, B. tenOever, G. C. Sen, S. Balachandran, G. N. Barber, R. Lin, J. Hiscott, Transcriptional profiling of interferon regulatory factor 3 target genes: direct involvement in the regulation of interferon-stimulated genes. J Virol 76, 5532-5539 (2002).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising (i) STING/TLR4 agonist nanoparticles (NPs), (ii) a MEK inhibitor, and (iii) a CDK4/6 inhibitor, in a pharmaceutically acceptable carrier.

2. The composition of claim 1, wherein the MEK inhibitor is trametinib, and the CDK4/6 inhibitor is palbociclib.

3. The composition of claim 1, wherein the STING/TLR4 agonists comprise cyclic-diguanosine monophosphate (cdGMP) and monophosphoryl-lipid A (MPLA).

4. The compositions of claim 1, wherein the NPs comprise DOPC (1,2-dioleoyl-sn-glycero-3-phophocholine), DSPC (1,2-distearoyl-sn-glycero-3-phosphocholine), cholesterol, and a PEG-conjugated lipid.

5. The composition of claim 4, wherein the PEG-conjugated lipid is PEG-DSPE.

6. The composition of claim 1, wherein the NPs further comprise a targeting moiety on the surface of the NPs.

7. A method of treating cancer in a subject, the method comprising administering a therapeutically effective amount of (i) STING/TLR4 agonist nanoparticles (NPs) and (ii) a combination of a MEK inhibitor and a CDK4/6 inhibitor.

8. The method of claim 7, wherein the MEK inhibitor is trametinib, and the CDK4/6 inhibitor is palbociclib.

9. The method of claim 7, wherein the STING/TLR4 agonists comprise cyclic-diguanosine monophosphate (cdGMP) and monophosphoryl-lipid A (MPLA).

10. The method of claim 7, wherein the NPs comprise DOPC (1,2-dioleoyl-sn-glycero-3-phophocholine), DSPC (1,2-distearoyl-sn-glycero-3-phosphocholine), cholesterol, and a PEG-conjugated lipid.

11. The method of claim 10, wherein the PEG-conjugated lipid is PEG-DSPE.

12. The method of claim 7, wherein the NPs further comprise a targeting moiety on the surface of the NPs.

13. The method of claim 7, wherein the cancer is lung cancer or pancreatic ductal adenocarcinoma (PDAC).

14. The method of claim 7, wherein the MEK inhibitor and CDK4/6 inhibitor are administered before the STING/TLR4 agonist nanoparticles (NPs), or concurrently with the STING/TLR4 agonist nanoparticles (NPs).

15. The method of claim 7, comprising administering the composition of claim 1.

16. STING/TLR4 agonist nanoparticles (NPs) and a combination of a MEK inhibitor and CDK4/6 inhibitor, for use in a method of treating cancer in a subject.

17. The composition of claim 1, for use in a method of treating cancer in a subject.

18. The STING/TLR4 agonist NPs and the combination of a MEK inhibitor and CDK4/6 inhibitor for use according to claim 16, wherein the cancer is a lung cancer or pancreatic ductal adenocarcinoma (PDAC).

19. The STING/TLR4 agonist NPs and the combination of a MEK inhibitor and CDK4/6 inhibitor for use according to claim 16, wherein the MEK inhibitor is trametinib, and the CDK4/6 inhibitor is palbociclib.

20. The STING/TLR 4 agonist NPs and the combination of a MEK inhibitor and CDK4/6 inhibitor for use according to claim 16, wherein the STING/TLR4 agonists comprise cyclic-diguanosine monophosphate (cdGMP) and monophosphoryl-lipid A (MPLA).

21. The STING/TLR4 agonist NPs and the combination of a MEK inhibitor and CDK4/6 inhibitor for use according to claim 16, wherein the NPs comprise DOPC (1,2-dioleoyl-sn-glycero-3-phophocholine), DSPC (1,2-distearoyl-sn-glycero-3-phosphocholine), cholesterol, and a PEG-conjugated lipid.

22. The STING/TLR4 agonist NPs and the combination of a MEK inhibitor and CDK4/6 inhibitor for use according to claim 21, wherein the PEG-conjugated lipid is PEG-DSPE.

23. The STING/TLR4 agonist NPs and the combination of a MEK inhibitor and CDK4/6 inhibitor for use according to claim 16, wherein the NPs further comprise a targeting moiety on the surface of the NPs.

24. The composition of claim 4, wherein the NPs further comprise DOPG (1,2-dioleoyl-sn-glycero-3-phospho-(1′-rac-glycerol)).

25. The composition of claim 5, wherein the PEG-DSPE is mPEG2000-DSPE (methoxy-poly(ethyleneglycol)-2000 1,2-distearoyl-sn-glycero-3-phophoethanolamine-N) or amine-PEG 2000-DSPE (amine-poly(ethylene glycol)-2000 1,2-distearoyl-sn-glycero-3-phophoethanolamine-N).

26. The composition of claim 6, wherein the targeting moiety comprises an ECM peptide or an EGFR peptide.

27. The method of claim 10, wherein the NPs further comprise DOPG (1,2-dioleoyl-sn-glycero-3-phospho-(1′-rac-glycerol)).

28. The method of claim 11, wherein the PEG-DSPE is mPEG2000-DSPE (methoxy-poly(ethyleneglycol)-2000 1,2-distearoyl-sn-glycero-3-phophoethanolamine-N) or amine-PEG 2000-DSPE (amine-poly(ethylene glycol)-2000 1,2-distearoyl-sn-glycero-3-phophoethanolamine-N).

29. The method of claim 12, wherein the targeting moiety comprises an ECM peptide or an EGFR peptide.

30. The method of claim 13, wherein the lung cancer or PDAC is a KRAS mutant lung cancer or a KRAS mutant PDAC.

31. The STING/TLR4 agonist NPs and the combination of a MEK inhibitor and CDK4/6 inhibitor for use according to claim 18, wherein the lung cancer or PDAC is a KRAS mutant lung cancer or a KRAS mutant PDAC.

32. The STING/TLR4 agonist NPs and the combination of a MEK inhibitor and CDK4/6 inhibitor for use according to claim 21, wherein the NPs further comprise DOPG (1,2-dioleoyl-sn-glycero-3-phospho-(1′-rac-glycerol)).

33. The STING/TLR4 agonist NPs and the combination of a MEK inhibitor and CDK4/6 inhibitor for use according to claim 22, wherein the PEG-DSPE is mPEG2000-DSPE (methoxy-poly(ethyleneglycol)-2000 1,2-distearoyl-sn-glycero-3-phophoethanolamine-N) or amine-PEG 2000-DSPE (amine-poly(ethylene glycol)-2000 1,2-distearoyl-sn-glycero-3-phophoethanolamine-N).

34. The STING/TLR4 agonist NPs and the combination of a MEK inhibitor and CDK4/6 inhibitor for use according to claim 23, wherein the targeting moiety comprises an ECM peptide or an EGFR peptide.
